SEQUENCE LISTING (13 sequences)

SEQ ID NO: 1 - DNA, 26 bp, Artificial sequence
  Synthetic primer
garcgcaarrgngcngcngmngargt

SEQ ID NO: 2 - DNA, 20 bp, Artificial sequence
  Synthetic primer
tcraartangtdatraartc

SEQ ID NO: 3 - DNA, 22 bp, Artificial sequence
  Synthetic primer
tygavaarcayaaggahaavgt

SEQ ID NO: 4 - DNA, 20 bp, Artificial sequence
  Synthetic primer
gagtggtavaryttvcgytt

SEQ ID NO: 5 - DNA, 26 bp, Artificial sequence
  Synthetic primer
gancgnaanngngcngcngnngangt

SEQ ID NO: 6 - DNA, 20 bp, Artificial sequence
  Synthetic primer
tcnaantangtnatnaantc

SEQ ID NO: 7 - DNA, 26 bp, Artificial sequence
  Synthetic primer
gaacgcaaaagngcngcngangaagt

SEQ ID NO: 8 - DNA, 26 bp, Artificial sequence
  Synthetic primer
garcgcaarrgngcngcngmngargt

SEQ ID NO: 9 - DNA, 20 bp, Artificial sequence
  Synthetic primer
tcraartangtdatraartc

SEQ ID NO: 10 - DNA, 26 bp, Artificial sequence
  Synthetic primer
gancgnaanngngcngcngnngangt

SEQ ID NO: 11 - DNA, 20 bp, Artificial sequence
  Synthetic primer
tcnaantangtnatnaantc

SEQ ID NO: 12 - DNA, 11 bp, Artificial sequence
  Synthetic primer
gcgcggaattc

SEQ ID NO: 13 - DNA, 12 bp, Artificial sequence
  Synthetic primer
gcgcgcaagctt

(19) United States
(12) Patent Application Publication
(10) Pub. No.: US 2009/0068650 A1
(43) Pub. Date: Mar. 12, 2009

(54) Metabolic Primers for the Detection of (Per) Chlorate-Reducing Bacteria and Methods of Use Thereof

(75) Inventors: Laurie Achenbach, Carbondale, IL (US); Kelly S. Bender, Pickneyville, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: SOUTHERN ILLINOIS UNIVERSITY, Carbondale, IL (US)

(21) Appl. No.: 11/815,654

(22) PCT Filed: Feb. 11, 2005
(86) PCT No.: PCT/US2005/004657

§ 371 Date: Oct. 16, 2008

(51) Int. Cl. C12Q 1/68; C07H 21/04; C40B 40/06

(52) U.S. Cl. 435/6; 536/24.33; 506/16

(57) ABSTRACT

The present invention is directed to metabolic primers for the detection of (per)chlorate-reducing bacteria and methods and compositions for use of the same in environmental bioremediation.

Certain aspects of the studies described herein were Federally-funded with the support of grant # DACA72-00-C-0016 from the Department of Defense.

### BACKGROUND

1. Field of the Invention

This invention relates to bioremediation of contaminants in the environmental samples, including for example, contamination in particulates such as soil and also in fluids such as groundwater. More particularly, the present invention is directed to methods and compositions for the detection of perchlorate-reducing bacteria at target decontamination site.

2. Background of the Related Art

Chemical contamination of the environment, particularly of soil and groundwater, is a widespread problem throughout the industrialized world. Industrial pollution has contaminated millions of acres of soil and associated aquifers. Often, cleanup of the contamination is hindered because the cost of remediation is significant. Moreover, many of the remediation techniques create additional problems which cause the land to remain unused or abandoned.

Recently, widespread perchlorate contamination of drinking water wells throughout the United States and especially throughout the southwest has become a significant cause for concern. Perchlorate contamination of ground and surface waters originates from, and is a direct effect of, unregulated ammonium perchlorate disposal practices from 1950 to 1997 (Renner et al., Environ. Sci. Technol. News 32:210 A, 1998). Ammonium perchlorate is an oxidant that is widely used in the aerospace, munitions, and fireworks industries. Widespread contamination has been documented in the waterways of California, and at least 19 other states in the United States. Similar contaminations have been reported in other countries that have aerospace, munitions and fireworks industries.

Perchlorate has been linked to a number of problems in human health. Excessive intake of perchlorate blocks iodine uptake and inhibits thyroid function and production of thyroid hormones, in addition, gastrointestinal irritation and skin rash, and hematological effects including agranulocytosis and lymphadenopathy have also been observed. In addition, it has been established that there is neurodevelopmental toxicity associated with perchlorate ingestion. As a result of these significant health concerns, drinking water utilities have begun monitoring and reporting perchlorate levels to the State agencies. In some states the Health Services Departments have set maximum limits on the amount of perchlorate in drinking water; this figure is typically in the order of 18 parts per billion (ppb) in order to minimize the risks to human health. The Environmental Protection Agency has established a provisional reference dose (“RFD”) of 14 mg of perchlorate per kg of body weight per day. Practical and efficient methods to treat water contaminated by perchlorate are needed to insure a safe drinking water supply in many communities.

Current methods of perchlorate remediation rely on the use of ion exchange resins to sequester perchlorate ions. (see e.g., U.S. Pat. No. 6,059,975). Conventional perchlorate-removal ion exchange resins have low selectivity coefficients and as such these resins are capable of loading only a few kilograms of perchlorate per cubic meter of removal resin. This produces a waste mass of loaded resin that must be disposed through, e.g., incineration. Disposal costs for these resins are therefore prohibitive because of the bulk volume of loaded to be disposed relative to the amount of perchlorate removed from the environmental target site. Other methods of perchlorate removal are actively being pursued, with bioremediation technologies emerging as a cost-effective and less-invasive alternative to physical or chemical practices (Urbansky et al., Biorem. J.:81-95, 1998).

Natural attenuation of perchlorate is a cost-effective alternative to current methods of perchlorate remediation. Such natural attenuation systems have been used in bioremediation of other contaminants and include the use of microbial populations to accelerate the breakdown of solids and the various contaminants associated with waste water. Such microbes are permitted to act upon the waste water or contaminated soils and they act to remove the pollutants faster than if nothing were used, and do so without the hazards and difficulties associated with chemical treatment.

The success of natural perchlorate remediation is dependent on the presence and activity of dissimilatory (per)chlorate-reducing bacteria (DPRB) within the target site that is undergoing remediation. Within the last 7 years, more than 40 different strains of dissimilatory (per)chlorate-reducing bacteria (DPRB) have been isolated from a diverse range of environments (Bruce et al., Environ. Microbiol. 1:319-329., 1999; Coates et al., Appl. Environ. Microbiol. 65:5234-5241., 1999; Kim et al., Water Res. 35:3071-3076., 2001; Logan et al., Appl. Environ. Microbiol. 67:2499-2506, 2001, 18, Rikken et al., Appl. Microbiol. Biotechnol., 45:420-426, 1996; Wallace et al., J. Ind. Microbiol. 16:68-72, 1996). Because of the metabolic capability and ubiquity of DPRB (Coates et al., Appl. Environ. Microbiol. 65:5234-5241., 1999), natural attenuation of perchlorate is garnering more and more interest. While studies by various groups have shown the ability of microbes to remediate perchlorate under environmental conditions (Hunter, Curr. Microbiol. 45:287-292., 2002; Kim et al., Water Res. 35:3071-3076., 2001; Tipton et al., J. Environ. Qual. 32:40-46, 2003), a quick, reliable method for detecting the presence and effectiveness of DPRB is needed to determine the natural attenuation candidacy of a contaminated site as well as for monitoring active degradation.

Traditionally, contaminant site evaluation for the presence of DPRB has been performed using labor-intensive enumeration and isolation techniques. However, it is well known that cultivation techniques are time-consuming and often prove unsuccessful in isolating the target bacteria due to both media selectivity and organism culturability (Dunbar et al., Appl. Environ. Microbiol. 63:1326-1331, 1997; Kaeberlein et al., Science, 296:1127-1129, 2002). To alleviate the limitations of cultivation-based methods, molecular techniques using the 16S rRNA gene have been employed to examine bacterial diversity in the environment (Aman et al., Microbiol. Rev. 59:143-169, 1995, Olsen et al., Annu. Rev. Microbiol. 40:337-365, 1986), and numerous primer sets have been developed for the 16S rRNA gene that target specific groups of bacteria. However, due to the fact that there is significant phylogenetic diversity of DPRB and because of their close phylogenetic relationships to non-(per)chlorate-reducing relatives, detection of DPRB using 16S ribosomal DNA (rDNA) primers is not recommended (Achenbach et al., Int. J. Syst. Evol. Microbiol. 51:527-533, 2001). As such, there remains a need to identify a more inclusive approach to the detection of DPRB that would allow an efficient molecular identification technique that will facilitate the rapid identification of the presence of DPRB at a given target site and/or allow prediction of whether a given target site is capable of undergoing perchlorate remediation.

### SUMMARY OF THE INVENTION

The present invention is directed to methods and compositions for use in bioremediation-related applications. Detection of (per)chlorate reducing bacteria is facilitated by the present invention, which in one aspect provides a composition comprising a first primer and a second primer, wherein the first primer has a nucleic acid sequence that comprises a sequence of SEQ ID NO:1 or SEQ ID NO:8 and the second primer has a nucleic acid sequence that comprises a sequence of SEQ ID NO:2 or SEQ ID NO:9, wherein the first and second primers are capable of hybridizing to a chlorite dismutase (cld) gene.

In additional embodiments, the composition may further comprise a third primer and a fourth primer, wherein the third primer has a nucleic acid sequence that comprises a sequence of SEQ ID NO:3 and the fourth primer has a nucleic acid sequence that comprises a sequence of SEQ ID NO:4, wherein the third and fourth primers are capable of hybridizing to a cld gene.

In an alternative embodiment, the composition is one in which there is a third primer and a fourth primer, wherein the third primer has a nucleic acid sequence that comprises a sequence of SEQ ID NO:5 or SEQ ID NO:10 and the fourth primer has a nucleic acid sequence that comprises a sequence of SEQ ID NO:6 or SEQ ID NO:11, wherein the third and fourth primers are capable of hybridizing to a cld gene.

In other specific embodiments, there is a composition that comprises a first primer and a second primer, wherein the first primer has a nucleic acid sequence that comprises a sequence of SEQ ID NO:3 and the second primer has a nucleic acid sequence that comprises a sequence of SEQ ID NO:4, wherein the first and second primers are capable of hybridizing to a chlorite dismutase (cld) gene. In other embodiments, such a composition may further comprise a third primer and a fourth primer, wherein the third primer has a nucleic acid sequence that comprises a sequence of SEQ ID NO:5 or SEQ ID NO:10 and the fourth primer has a nucleic acid sequence that comprises a sequence of SEQ ID NO:6 or SEQ ID NO:11, wherein the third and fourth primers are capable of hybridizing to a cld gene.

Additional aspects contemplate compositions that further comprise a fifth primer and a sixth primer, such that in addition to SEQ ID NO:1 or 8, SEQ ID NO:2 or 9, SEQ ID NO:3, SEQ ID NO:4, the composition further comprises a fifth primer that has a nucleic acid sequence that comprises a sequence of SEQ ID NO:5 or SEQ ID NO:10 and the sixth primer has a nucleic acid sequence that comprises a sequence of SEQ ID NO:6 or SEQ ID NO:11, wherein the fifth and sixth primers are capable of hybridizing to a cld gene.

Preferably, each of the first, second, third, fourth, fifth and sixth primers each independently comprise between 20 and 30 nucleotide bases in length. Other preferred embodiments contemplate that the first, second, third, fourth, fifth and sixth primers each independently comprise between 20 and 40 nucleotide bases in length. Still additional embodiments contemplate that each of the first, second, third, fourth, fifth and sixth primers each independently comprise between 20 and 50 nucleotide bases in length.

In specific aspects of the invention, it is contemplated that the cld gene is from dissimilatory (per)chlorate-reducing bacteria (DPRB) species. DPRB species are well known to those of skill in the art, and simply by way of example, include, but are not limited to a bacterium from the Dechloromonas spp., Azoarcus spp., Dechlorospirillum spp., Dechloromarinus spp., Ideonella spp., Magnetospirillum spp., Pseudomonas spp., Rhodocyclus spp., Rhodospirillum spp., Azospirillum spp., Wolinella spp., Xanthomonas spp. In specific embodiments, the DPRB is selected from the group consisting of Dechloromonas agitate, Dechloromonas aromatica, Azospira suillum, Dechlorospirillum anomalous, Dechloromarinus chlorophilus, Ideonella dechloratans, and Magnetospirillum magnetotacticum.

In specific embodiments, the primer is detectably labeled.

Also contemplated herein is an oligonucleotide primer pair wherein the first primer of the primer pair comprises a sequence of SEQ ID NO:1 or SEQ ID NO:8 and the second primer of the primer pair comprises a sequence of SEQ ID NO:2 or SEQ ID NO:9. Other preferred oligonucleotide primer pairs are those in which the first primer of the primer pair comprises a sequence of SEQ ID NO:3 and the second primer of the primer pair comprises a sequence of SEQ ID NO:4. Yet further preferred primer pairs include those in which first primer of the primer pair comprises a sequence of SEQ ID NO:5 or SEQ ID NO:10 and the second primer of the primer pair comprises a sequence of SEQ ID NO:6 or SEQ ID NO:11. Preferably, in such compositions, at least one of the primers is detectably labeled.

Other aspects specifically contemplated an oligonucleotide primer which has the nucleotide sequence defined in any one of SEQ ID NOs: 1, 2, 3, 4, 5, 6, 8, 9, 10, or 11. Such an oligonucleotide may be between 20 and 50 nucleotide bases. Preferably, the oligonucleotide primer is detectably labeled. The label may be any label that is conventionally used to facilitate detection of the primer or the product of the reaction in which the primer is used. For example, the primer is labeled with an epitope, fluorophore, metal particle, enzyme, carbohydrate, polypeptide, radioactive isotope, dye, biotin, or digitonin.

The oligonucleotide primer pairs discussed above may be such that at least on the oligonucleotides in the pair is labeled with an epitope, fluorophore, metal particle, enzyme, carbohydrate, polypeptide, radioactive isotope, dye, biotin, or digitonin.

The present invention particularly contemplates methods of detecting the presence of (per)chlorate reducing bacteria in a sample comprising subjecting DNA of bacterial cells in the sample to a first polymerase chain reaction amplification using a first pair of primers (e.g., a pair of primers of in which the first primer comprises a sequence of SEQ ID NO:1 or SEQ ID NO:8 and the second primer comprises a sequence of SEQ ID NO:2 or SEQ ID NO:9); and detecting the product or products of the first polymerase chain reaction amplification, thereby identifying the presence of the (per)chlorate-reducing bacteria in the sample. In additional embodiments, the method may advantageously further comprise subjecting the DNA to a second polymerase chain reaction amplification using a second pair of primers (e.g., a pair of primers in which the first primer comprises a sequence of SEQ ID NO:3 and the second primer comprises a sequence of SEQ ID NO:4); and detecting the product or products of the second polymerase chain reaction amplification thereby identifying the presence of the (per)chlorate-reducing bacteria in the sample.

Other methods of the invention involve detecting the presence of (per)chlorate reducing bacteria in a sample comprising subjecting DNA from the sample to a first polymerase chain reaction amplification using a pair of primers in which the first primer comprises a sequence of SEQ ID NO:3 and the second primer comprises a sequence of SEQ ID NO:4; and detecting the product or products of the first polymerase chain reaction amplification, thereby identifying the presence of the (per)chlorate-reducing bacteria in the sample.

Also contemplated herein are methods of detecting the presence of (per)chlorate-reducing bacteria in a sample comprising subjecting DNA from the sample to a first polymerase chain reaction amplification using a first pair of primers (e.g., a pair of primers of in which the first primer comprises a sequence of SEQ ID NO:1 or SEQ ID NO:8 and the second primer comprises a sequence of SEQ ID NO:2 or SEQ ID NO:9); isolating the amplification products from such a reaction; using those isolated amplification products as a template for a second polymerase chain reaction amplification using a primer pair in which the first primer comprises a sequence of SEQ ID NO:3 and the second primer comprises a sequence of SEQ ID NO:4; and ultimately detecting the product or products of the second polymerase chain reaction amplification, thereby identifying the presence of the (per)chlorate-reducing bacteria in the sample.

Another method of the invention comprises detecting the presence of (per)chlorate-reducing bacteria in a sample comprising subjecting DNA from the sample to a first polymerase chain reaction amplification using a first pair of primers first pair of primers (e.g., a pair of primers of in which the first primer comprises a sequence of SEQ ID NO:5 or SEQ ID NO: 10 and the second primer comprises a sequence of SEQ ID NO:6 or SEQ ID NO:11; isolating the amplification products from such a reaction; using those isolated amplification products as a template for a second polymerase chain reaction amplification using a primer pair in which the first primer comprises a sequence of SEQ ID NO:3 and the second primer comprises a sequence of SEQ ID NO:4; and detecting the product or products of the second polymerase chain reaction amplification, thereby identifying the presence of the (per)chlorate-reducing bacteria in the sample.

In the above methods it may be desirable that the DNA is isolated from bacterial lysates from the sample prior to the first polymerase chain reaction.

The above methods may be particularly suited to the analysis of contaminated environmental samples. In specific embodiments; the sample used in the methods is a water sample. In other embodiments, the sample is a soil sample. In other embodiments, the water sample is collected from a water supply that has been contaminated with perchlorate. Alternatively, the soil sample is collected from land that has been contaminated with perchlorate.

The perchlorate contamination may be from any contaminating source, including e.g., a result of waste disposal from paper mill waste, airbag production, firework manufacture and use, fertilizer manufacture and use.

Also provided herein are methods of determining whether a bioremediation formulation that comprises bacteria will be effective at decreasing (per)chlorate contamination comprising subjecting DNA from the bioremediation formulation to a first polymerase chain reaction amplification using a first pair of primers (e.g., a pair of primers of in which the first primer comprises a sequence of SEQ ID NO:1 or SEQ ID NO:8 and the second primer comprises a sequence of SEQ ID NO:2 or SEQ ID NO:9; and detecting the product or products of the first polymerase chain reaction amplification, wherein the presence of the amplification products indicates the presence of (per)chlorate reducing bacteria in bioremediation formulation thereby indicating that the formulation is effective at reducing perchlorate contamination. In other aspects of the invention, the above method may further comprising subjecting the DNA to a second polymerase chain reaction amplification using a pair of primers of SEQ ID NO:3 and SEQ ID NO:4; and detecting the presence of (per)chlorate-reducing bacteria in the bioremediation formulation by visualizing the product or products of the second polymerase chain reaction amplification, wherein the presence of the amplification products indicates that the formulation is effective at reducing perchlorate contamination.

The methods described herein may also be used in determining whether a bioremediation formulation that comprises bacteria will be effective at decreasing (per)chlorate contamination comprising subjecting the DNA from the bioremediation formulation to a first polymerase chain reaction amplification using a pair of primers of claim in which the first primer has a sequence of SEQ ID NO:3 and the second primer has a sequence of SEQ ID NO:4; and detecting the (per)chlorate-reducing bacteria by visualizing the product or products of the first polymerase chain reaction amplification.

Further methods involve determining whether a bioremediation formulation that comprises bacteria will be effective at decreasing (per)chlorate contamination comprising subjecting the DNA from the bioremediation formulation to a first polymerase chain reaction amplification using a first pair of primers (e.g., a pair of primers of in which the first primer comprises a sequence of SEQ ID NO:1 or SEQ ID NO:8 and the second primer comprises a sequence of SEQ ID NO:2 or SEQ ID NO:9; isolating the amplification products from that reaction; using those isolated amplification products as a template for a second polymerase chain reaction amplification using a primer pair of SEQ ID NO:3/SEQ ID NO:4; and detecting the (per)chlorate-reducing bacteria by visualizing the product or products of the second polymerase chain reaction amplification.

Another method is directed to determining whether a bioremediation formulation that comprises bacteria will be effective at decreasing (per)chlorate contamination comprising subjecting the DNA from the bioremediation formulation to a first polymerase chain reaction amplification using a first pair of primers (e.g., a pair of primers of in which the first primer comprises a sequence of SEQ ID NO:5 or SEQ ID NO:10 and the second primer comprises a sequence of SEQ ID NO:6 or SEQ ID NO:11; isolating the amplification products from such a reaction; using the isolated amplification products as a template for a second polymerase chain reaction amplification using a primer pair of SEQ ID NO:3/SEQ ID NO:4; and detecting the (per)chlorate-reducing bacteria by visualizing the product or products of the second polymerase chain reaction amplification.

In such methods, the DNA is preferably isolated from a bacterial lysate from the bioremediation formulation prior to the initial first polymerase chain reaction amplification step. In specific embodiments, the bioremediation formulation is a cocktail of microorganisms that are used to remove contaminants from a sample of soil or water in need of decontamination, wherein the cocktail of microorganisms comprises a mixture of DPRBs. In preferred embodiments, the cocktail of microorganisms further microorganisms that are denitrifiers. Preferably, the cocktail of microorganisms further comprises microorganisms that can degrade toluene, xylene, benzene, petroleum, and creosote.

Also contemplated herein is a method of determining whether a sample contains bacteria that is reducing (per)chlorate in the sample comprising isolating nucleic acid from the sample; incubating the nucleic acid with a DNase to isolate RNA; performing a reverse transcription reaction on the RNA using one or more of the primers selected from the group consisting of SEQ ID NO:1, 2, 3, 4, 5, 6, 8, 9, 10 and 11; and detecting the product or products of the reverse transcription reaction that are expressing chlorite dismutase, thereby identifying the presence of bacteria in the sample that are expressing chlorite dismutase for reducing the (per)chlorate content of the sample. This method may further comprise isolating the reaction products from the reverse transcription reaction and using the isolated reaction products as a template for a polymerase chain reaction amplification using a primer pair in which the first primer comprises a sequence of SEQ ID NO:1 or SEQ ID NO:8 and the second primer of the primer pair comprises a sequence of SEQ ID NO:2 or SEQ ID NO:9 and/or performing a polymerase chain reaction amplification using a primer pair in which the first comprises a sequence of SEQ ID NO:3 and the second primer of the primer pair comprises a sequence of SEQ ID NO:4.

The invention is further directed to a method of determining whether a sample contains bacteria that is reducing (per)chlorate in the sample comprising isolating nucleic acid from the sample; incubating the nucleic acid with a DNase to isolate RNA; performing a reverse transcriptase reaction on the RNA using one or more of the primers selected from the group consisting of SEQ ID NO:1, 2, 3, 4, 5, 6, 8, 9, 10 and 11; isolating the reaction products from the reaction; using the isolated reaction products as a template for a polymerase chain reaction amplification using a primer pair in which the first primer comprises a sequence of SEQ ID NO:3 and the second primer of the primer pair comprises a sequence of SEQ ID NO:4; and detecting the product or products of such a polymerase chain reaction amplification step, thereby identifying the presence of bacteria in the sample that are expressing chlorite dismutase for reducing the (per)chlorate content of the sample.

Also contemplated herein is a method of determining whether a sample contains bacteria that is reducing (per)chlorate in the sample comprising isolating nucleic acid from the sample; incubating the nucleic acid with a DNase to isolate RNA; performing a reverse transcriptase reaction on the RNA using one or more of the primers selected from the group consisting of SEQ ID NO:1, 2, 3, 4, 5, 6, 8, 9, 10 and 11; isolating the reaction products from that reverse transcriptase reaction; using the isolated reaction products as a template for a polymerase chain reaction amplification using a primer pair in which the first primer comprises a sequence of SEQ ID NO:1 or SEQ ID NO:8 and the second primer of the primer pair comprises a sequence of SEQ ID NO:2 or SEQ ID NO:9; and detecting the product or products of such a polymerase chain reaction amplification, thereby identifying the presence of bacteria in the sample that are expressing chlorite dismutase for reducing the (per)chlorate content of the sample. In certain embodiments, such a method may advantageously further comprise isolating the amplification products of the polymerase chain reaction prior to performing the detection step and using the isolated amplification products as a template for a polymerase chain reaction amplification using a primer pair in which the first primer comprises a sequence of SEQ ID NO:3 and the second primer comprises a sequence of SEQ ID NO:4.

The invention also is directed to kits for amplifying chlorite dismutase (cld) polynucleotide, the kit comprising: composition or an oligonucleotide pair or an oligonucleotide as described herein above, wherein the composition; and instructions for carrying out any one or more of the methods discussed above. Preferably, the kit may further comprise enzymes and nucleotide components of a PCR reaction. The kits of the invention also may comprise one or more solid supports. In certain embodiments, the primers described herein are preferably arranged as arrays on a solid support. Preferably, the arrays are addressable and/or detectable such that the skilled individual may readily detect the primer from a signal or from a specific “address” or location on the solid support. Other embodiments contemplated are those in which the primers are provided in separate containers.

Also provided herein is a library of primers for the detection of a cld gene from DPRB, the library comprising at least 6 primers derived from the sequences set forth in SEQ ID NO:1, 2, 3, 4, 5, 6, 8, 9, 10 and 11. Preferably, the primers are provided on an addressable array.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Amino acid alignment of cld gene products from D. agitata (D.agit), I. dechloratans (I.dech), M. magnetotacticum (M.magn), “D. aromatica” (D.arom), Dechloromonas sp. strain LT1 (DcmLT1), Pseudomonas sp. strain PK (Ps. PK), “D. chlorophilus” (D.chlo), D. suillum (D.suil), “D. anomalous” (D.anom), Dechlorospirillum sp. strain DB (st. DB), and strain CR (st. CR). Numbers correspond to residues from the D. agitata mature protein. “˜” denotes alignment gaps; “−” denotes unknown sequence data; “.” denotes identical residues. The DCD-F/DCD-R primer set targeted the lightly shaded regions, while the UCD-238F/UCD-646 primer set targeted the darkly shaded regions.

FIG. 2 Amplification of a 484-bp internal region of the cld gene using the DCD-F/DCD-R primer set. Lane 1, “D. aromatica”; lane 2, M. magnetotacticum; lane 3, I. dechloratans; lane 4, Pseudomonas sp. strain PK; lane 5, D. suillum; lane 6, “D. chlorophilus”; lane 7, “D. anomalous”; lane 8, D. strain LT1; lane 9, negative control (no DNA); lane 10, 1-kb ladder.

FIG. 3 Amplification of a 408-bp internal region of the cld gene using the UCD-238F/UCD-646R primer set. Lane 1, D. agitata; lane 2, “D. aromatica”; lane 3, M. magnetotacticum; lane 4, I. dechloratans; lane 5, Pseudomonas sp. strain PK; lane 6, D. suillum; lane 7, “D. chlorophilus”; lane 8, “D. anomalous”; lane 9, Dechloromonas species strain LT-1; lane 10, R. tenuis; lane 11, P. stutzeri; lane 12, Escherichia coli; lane 13, negative control (no DNA); lane 14, 100-bp ladder.

FIG. 4. Testing of the universal cld gene primer sets on environmental DNAs. Top of gel, touchdown PCR using the DCD-F/DCD-R primer set, corresponding to a 484-bp internal region of the cld gene. Bottom of gel, nested PCR on the above reactions, using the UCD-238F/UCD-646R primer set, corresponding to a 408-bp internal region of the cld gene. Lane 1, Pseudomonas sp. strain PK (positive control); lane 2, Los Alamos well 2; lane 3, Los Alamos well 3; lane 4, Los Alamos well 4; lane 5, Los Alamos well 5; lane 6, Los Alamos well 7; lane 7, campus library pond; lane 8, campus library soil; lane 9, campus lake; lane 10, Lake Fryxell sediment; lane 11, Lake Fryxell 7-m water column; lane 12, Lake Fryxell 12-m water column; lane 13, Lake Hoare 12-m water column; lane 14, Lake Hoare mat; lane 15, Vida; lane 16, negative control (no DNA); lane 17, 100-bp ladder.

FIGS. 5A and 5B. cld and 16S rDNA phylogenetic trees. (FIG. 5A) cld phylogenetic tree generated from an alignment of 369 bp. (FIG. 5B) 16S rDNA phylogenetic tree generated from an alignment of 1,424 bp. The numbers correspond to bootstrap values from 100 replicates. D. agitata (Dcm.agit), “D. aromatica” (Dcm.arom), D. strain LT1 (Dcm.LT1), D. suillum (Dcs. suil), “D. anomalous” (Dsp.anom), “D. chlorophilus” (Dma.chlo), I. dechloratans (I.dech), M. magnetotacticum (M.magn), Pseudomonas sp. strain PK (Pseud.PK), Dechlorospirillum sp. strain DB (DB), and strain CR(CR) were analyzed.

### DESCRIPTION OF THE PREFERRED EMBODIMENTS

There are significant environmental problems associated with the presence of perchlorate in the ground water and soil compositions of areas where there has previously been significant activities from the aerospace, munitions, and fireworks industries. These industries have produced significant quantities of ammonium perchlorate and the unchecked disposal of this compound over the last 5 decades has resulted in the contamination the ground and surface waters throughout the United States and other countries (Renner et al., Environ. Sci. Technol. News 32:210A, 1998). Remediation of soils and water to remove the perchlorate contaminants has typically involved resin-based ion exchange chromatography, which is an inefficient method because of the bulk of resin required and the fact that the perchlorate-loaded resin ultimately must be disposed.

Biological methods for remediation or clean-up of the environment are compelling in that they employ natural organisms. Microorganisms have been used to clean-up oil spills, sewage effluence, chlorinated solvents, pesticides and the like. The ability of microorganisms to remove the contaminants from a contaminated site is nature's way of cleaning-up the environment. In the present application, there are provided methods and compositions for determining the presence and efficacy of one such set of microorganisms.

DPRB are excellent microbial bioremediators in that they are able to live in a diverse range of environments and are able to metabolize (per)chlorate. As such, natural attenuation of (per)chlorate contaminants is of significant economic and environmental interest. The present invention specifically is directed to identification of the presence of these bacteria and compositions that may be used in the field to determine whether a given target site will undergo bioremediation naturally or whether additional intervention is required.

Metabolic primer sets have been applied to a variety of bioremediative studies for the detection of specific bacteria. For example, since many denitrifiers are able to degrade toluene and xylene, Braker and colleagues developed primer sets targeting two nitrate reductase genes that allowed for the qualitative detection of denitrifiers in the environment (Braker et al., Appl. Environ. Microbiol., 64:3769-3775, 1998). And while primers for the catechol 2,3-dioxygenase were used to detect bacteria capable of aerobically degrading benzene, toluene, and xylene, they were also used in quantitative PCR to show an increase in gene copy number after soil samples were amended with petroleum (Mesearch et al., Appl. Environ. Microbiol. 66:678-683, 2000). In specific embodiments, the invention is directed to primers that are able to target a gene that is essential to the metabolic pathway of (per)chlorate reducing bacteria but do not target their close phylogenetic non-(per)chlorate-reducing relatives. These primers allow for the rapid, sensitive and inexpensive identification of presence of DPRB. Certain aspects of the invention are described in further detail herein below.

An ideal target gene for the environmental detection of DPRB is the chlorite dismutase gene, cld. This is based on previous studies indicating that chlorite dismutation is essential to the (per)chlorate reduction pathway (Bruce et al., Environ. Microbiol. 1:319-329., 1999; Coates et al., Appl. Environ. Microbiol. 65:5234-5241., 1999; van Ginkel et al., Arch. Microbiol. 166:321-326, 1996). To date, no other enzyme has been isolated that is capable of converting chlorite to oxygen and chloride ions. Hybridization analysis using a chlorite dismutase immunoprobe indicated that all DPRB tested possess the chlorite dismutase enzyme. Moreover, the chlorite dismutase antibody did not bind to close non-(per)chlorate reducing relatives (O'Connor et al., Appl. Environ. Microbiol. 68:3108-3113, 2002). Similarly, a DNA probe targeting the cld gene only hybridized to genomic DNA (gDNA) from DPRB and the non-(per)chlorate reducer Magnetospirillum magnetotacticum. The probe did not hybridize to any other close phylogenetic relatives incapable of (per)chlorate reduction (Bender et al., Appl. Environ. Microbiol. 68:4820-4826, 2002).

Thus, studies suggest that the chlorite dismutase gene is unique to and required by all DPRB. Thus, a metabolic primer set targeting this gene would be useful for the molecular detection of DPRB in the environment. However, the efficacy of this metabolic primer set is dependent upon regions of sequence conservation within the cld gene, information which is currently unavailable due to the paucity of cld gene sequences in the database. The primers of the present invention are designed to universally detect this gene. Hence, this gene is the target nucleic acid for the methods of the present invention. As used herein, the term “target nucleic acid” or “nucleic acid target” refers to a particular nucleic acid sequence of interest. The “target” can exist in the presence of other nucleic acid molecules or within a larger nucleic acid molecule. In the present invention, target nucleic acid is a sequence that is a chlorite dismutase gene from any DPRB. The DPRB may be present in any sample, including soil samples, groundwater samples, isolated populations of DPRBs, cocktails of bacterial populations used in bioremediation processes and the like. The inventors have developed the primers of the present invention such that they hybridize to the cld gene from DPRB. The results and discussion of the studies leading to the identification of these primers is provided in the Examples.

### A. PRIMERS, OLIGONUCLEOTIDES AND PRODUCTION THEREOF

The following section provides a discussion of preferred primer and oligonucleotide compositions of the invention. The term “nucleic acid” as used herein refers to a linear sequence of nucleotides (bases) linked to one another by a phosphodiester bond between 3′-position of a pentose of one nucleotide and 5′-position of a pentose of another nucleotide. The term “polynucleotide” refers to a nucleic acid including a sequence of nucleotides more than about 100 bases. The term “oligonucleotide” refers to a short polynucleotide or a portion of polynucleotide including about 2-100 bases.

As used herein a “primer” refers to an oligonucleotide, which is capable of acting as a point of initiation of nucleic acid synthesis when the primer is placed under conditions in which synthesis of a primer extension product that is complementary to a nucleic acid strand is induced, i.e. in the presence of nucleotides and an inducing agent such as DNA polymerase and at a suitable temperature and pH. The primer may be a naturally occurring oligonucleotide or may be a purified restriction digest or produced synthetically. The oligonucleotide primers may be used, for example, in a PCR method as a primer for polymerization, in specific embodiments, the same primer may be in reverse transcription reaction in which the enzyme catalyzing the polymerization is a reverse transcriptase. Herein, oligonucleotides and primers of the invention may contain some modified linkages such as a phosphorothioate bond. The primers may also comprise a degenerate base, such as N base. Alternatively, one or more of the bases may be a universal base, such as e.g., hypoxanthine, as its ribo- or 2′-deoxyribonucleoside which is known for its ability to form base pairs with the other natural DNA/RNA bases. Nucleotide analog can be incorporated into the primers by methods well known in the art. The only requirement is that the incorporated nucleotide analog must serve to base pair with target polynucleotide sequences. For example, certain guanine nucleotides can be substituted with hypoxanthine, which base pairs with cytosine residues. Alternatively, adenine nucleotides can be substituted with 2,6-diaminopurine, which can form stronger base pairs than those between adenine and thymidine.

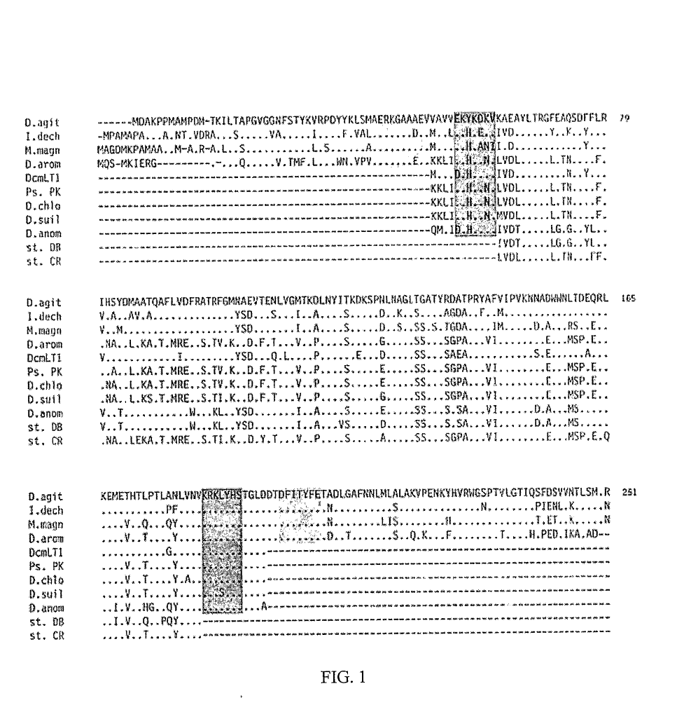

In exemplary embodiments of the present invention, primer sets targeting the chlorite dismutase gene were designed based on areas of amino acid and nucleotide sequence conservation. The primers of SEQ ID NO:1 and SEQ ID NO:2 were developed based on the amino acid conservation of the cld sequences from Dechloromonas agitata, Dechloromonas aromatica, Ideonella dechloratans, and M. magnetotacticum. The primers of SEQ ID NO:3 and SEQ ID NO:4 were developed from an expanded alignment that also included the cld gene sequences from Pseudomonas sp. strain PK, Dechloromonas sp. strain LT1, Azospira suillum, “Dechlorospirillum anomalous” strain WD, and “Dechloromarinus chlorophilus” sp. strain NSS. These alignments are shown in FIG. 1. Primers were synthesized by Integrated DNA Technologies, Coralville, Iowa.

The primers include the following:

| | |
|---|---|
| SEQ ID NO: 1: | [5′-GA(A/G)CGCAA(A/G)(A/G)GNGCNGCNG(A/C)NGA(A/G)GT-3′] |
| SEQ ID NO: 2: | [5′-TC(A/G)AA(A/G)TANGT(A/T/G)AT(A/G)AA(A/G)TC-3′] |
| SEQ ID NO: 3 | [5′-T(C/T)GA(A/C/G)AA(A/G)CA(C/T)AAGGA(A/T/C)AA(A/C/G)GT-3′] |
| SEQ ID NO: 4: | [5′-GAGTGGTA(A/C/G)A(A/G)(C/T)TT(A/C/G)CG(C/T)TT-3′] |
| SEQ ID NO: 5 | [5′-GANCGNAANNGNGCNGCNGNNGANGT-3′] |
| SEQ ID NO: 6 | [5′-TCNAANTANGTNATNAANTC-3′] |

Each of the above primers is a degenerate primer. Degenerate primers are useful for pulling out one part of a gene sequence when the gene sequence in related organisms is unknown. Degenerate primers are thus designed to match an amino acid sequence. Typically, one gathers sequences from a large range of organisms and translates them to amino acid sequence and aligns them. Based on these amino acid alignments, regions of the sequence that are highly conserved at the amino acid level are readily identified. These conserved regions become possible locations for degenerate primers. Preferably, at least 2 blocks of conserved amino acids should be present to enable the design of PCR primers. FIG. 1 herein shows the sequence alignments performed herein. A further alignment can be done at nucleotide level if desired.

Typically, the conserved regions chosen as the basis for the primer design should be at least 5 amino acids in length, more preferably 6, 7, 8, 9, 10 or more amino acids in length. As such, the primers should be 20-30 mer in length and it is preferred that the minimum size is 20 bases in length (i.e., a 20 mer). In certain embodiments, the efficiency of the degenerate primers may be increased by adding an oligonucleotide tail to the degenerate primers on the 5′ ends. This helps to increase the PCR efficiencies of these primers by increasing primer length and hence allows an increase in the annealing temperature. Although the tails do not help in the first few rounds of PCR when only the genomic template is being amplified, the tails do match in subsequent PCR cycles when the short PCR products containing the primers at each end are being amplified. Tails from commonly used restriction sites are particularly useful. For example a tail from an EcoRI site e.g., GCGCGGAATTC (SEQ ID NO:12) can be added to the 5′ end of the degenerate primer. Another useful tail, GCGCGCAAGCTT (SEQ ID NO:13), from the HindIII restriction site could be added to the 5′ end of a primer of the present invention.

Degeneracy of the primers depends on a multitude of factors including the template. 1000-10,000 fold degeneracy have been done. However the degeneracy can be lowered with the use of inosines for substituting 4 base wobbles instead of using all 4 base substitutions Thus, one or more of the “N” sequences in the above primers may be replaced with an inosine residue. To obtain the degeneracy of the primers, all the degeneracy values incorporated into the primer sequence are multiplied. Thus, other specific primers of the present invention include:

| | |
|---|---|
| SEQ ID NO: 8: | [5′-GA(A/G)CGCAA(A/G)(A/G)GIGCNGCIG(A/C)IGA(A/G)GT-3′] |
| SEQ ID NO: 9: | [5′-TC(A/G)AA(A/G)TAIGT(A/T/G)AT(A/G)AA(A/G)TC-3′] |
| SEQ ID NO: 10 | [5′-GAICGIAAIIGIGCIGCIGIIGAIGT-3′] |
| SEQ ID NO: 11: | [5′-TCIAAITAIGTIATIAAITC-3′] |

Of the above primers, SEQ ID NO:8 may be used instead of SEQ ID NO:1 herein throughout, SEQ ID NO:9 may be used instead of SEQ ID NO:2 herein throughout, SEQ ID NO:10 may be used instead of SEQ ID NO:5 herein throughout, and SEQ ID NO:11 may be used instead of SEQ ID NO:6 herein throughout.

In the primers described herein, according to standard nomenclature “N” refers to any one of the bases A, C, G, or T and it is presented at the third “wobble” position of the nucleic acid codons. Those of skill in the art could modify these primers by fixing one or more of the “N” residues in any of the primers with a specific nucleotide. It is specifically contemplated that the individual sequences derived from the degenerate primers in which the “N” is a set base are specifically part of the present invention. Simply by way of example, SEQ ID NO:1 may yield the primer: [5′-GAACGCAAAAGNGCNGCNGANGAAGT-3′] (SEQ ID NO:7) derived by fixing the “A/G” choice at position 3 of the oligonucleotide as “A,” fixing the “A/G” choice at position 9 of the oligonucleotide as “A”, fixing the “A/G” choice at position 10 of the oligonucleotide as “A”, fixing the “A/C” choice at position 20 of the oligonucleotide as “A”, fixing the “A/G” choice at position 24 of the oligonucleotide as “A”. In like manner, the other permutations of the above degenerate primers also can be readily determined. Each of these permutations is particularly part of this invention and the specific sequences have not been written out as individual primer sequence, simply for the purposes of clarity and not because they are excluded from the written description. One embodiment of the invention contemplates a library of primers that are generated from any one or more of the primers of SEQ ID NO:1 through SEQ ID NO:6 and SEQ ID NO:8 through SEQ ID NO:11. In certain exemplary embodiments, it may be desirable to array such a library of primers on a sequencing chip or other nucleic acid microarray.

Sequences of about 17 bases long should occur only once in the genome and, therefore, suffice to specify a unique target sequence. The primers of the present invention are typically of this size. As used herein, an oligonucleotide that “specifically hybridizes” to a given target nucleic acid (e.g., DNA; RNA) means that hybridization under suitably (e.g., high) stringent conditions allows discrimination of that target nucleic acid from other genes. Although shorter oligomers are easier to make, numerous other factors are involved in determining the specificity of hybridization. Both binding affinity and sequence specificity of an oligonucleotide to its complementary target increases with increasing length. It is contemplated that exemplary oligonucleotides of 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more base pairs can be prepared. In preferred aspects of the invention the oligonucleotide primers are between about 17 to about 50 bases in length and serve as primers in amplification reactions to amplify the sequence of any cld gene sequence from a DPRB sample. The oligonucleotides may include the degenerate primers (which may be anywhere from 17 to 50 bases in length) but may also include 5′ or 3′ flanking regions or tails to facilitate amplification of the products. In particular, as discussed above, 5′ tails on the degenerate primers are useful for increasing amplification efficiency. Thus, as used herein, the “primer” sequence is the degenerate primer discussed above, an oligonucleotide sequence may be the primer sequence alone, or it may be the primer sequence in addition to other nucleic acids.

The term “complementary” is used when defining a pair of nucleotide sequences, for example, a base pair of A/T or C/G, that match each other according to the base pairing rules. For example, a sequence of 5′-A-G-T-3′ is complementary to a sequence of 3′-T-C-A-5′. Nucleotide sequences may be “partially” or “perfectly” complementary to one another so that they form partially matching base pairs or perfectly matching base pairs.

Methods for the production of primers are well known to those of skill in the art as routine synthesis techniques. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Synthesis of modified oligonucleotides (e.g., oligonucleotides comprising 2′-O-methyl nucleotides and/or phosphorothioate, methylphosphonate, or boranophosphate linkages, e.g., for use as nuclease resistant primers) are described in e.g., Oligonucleotides and Analogs (1991), IRL Press, New York; Shaw et al. (1993), Methods Mol. Biol. 20:225-243; Nielsen et al. (1991), Science 254:1497-1500; and Shaw et al. (2000) Methods Enzymol. 313:226-257. Detailed procedures for the phospho-triester and hydrogen phosphonate methods of oligonucleotide synthesis are described in the U.S. Pat. No. 4,458,066.

Oligonucleotides, including modified oligonucleotides (e.g., oligonucleotides comprising fluorophores and quenchers, 2′-O-methyl nucleotides, and/or phosphorothioate, methylphosphonate, or boranophosphate linkages) can also be ordered from a variety of commercial sources known to persons of skill. There are many commercial providers of oligo synthesis services, and thus, this is a broadly accessible technology. Companies such as The Midland Certified Reagent Company (www.mcrc.com), The Great American Gene Company (www.genco.com), ExpressGen Inc. (www.expressgen.com), QIAGEN (http://oligos.qiagen.com) and many others provide a readily available commercial service for the synthesis of oligonucleotide and as such the primers and oligonucleotides of the present invention may be commercially synthesized once the identity of the oligonucleotides is provided herein. The probes used herein were prepared by Integrated DNA Technologies, (Coralville, Iowa).

The present invention requires the use of a probe or primer pairs that are specific for cld from a DPRB. To develop these probes or primers, one must first determine what genetic sequences are conserved between the many strains of DPRB. If one were to use a sequence derived from only a few strains, one would risk not detecting bacterial strain that had mutated slightly from this group.

The inventors compared the cld sequence from D. agitata, I. dechloratans, M. magnetotacticum, “D. aromatica”, Dechloromonas sp. strain LT1, Pseudomonas sp. strain PK, “D. chlorophilis”, D. suillum, “D. anomalous”, Dechlorospirillum sp. strain DB, and strain CR (shown in FIG. 1) and looked for highly conserved nucleotide sequences. As discussed above, the design of degenerate primers typically employs an amino acid sequence of at least 5 amino acids, which would produce a 15-mer, however, it is preferred that the primers should be greater than or equal to 20 nucleotides in length. Table 2 and FIG. 1 describes the amino acid sequences identities of cld proteins from various sources.

A probe of the invention suitable to hybridize with a cld gene sequence will be at least 20 nucleotides in length and may be chosen from the entire length of the cld sequence. The probe should preferably have a GC content of approximately 50%.

To derive primers from the cld sequences, one must first choose sequences that when amplified would produce a DNA segment of sufficient length. An exemplary product length is a DNA segment of at least 100 nucleotides. If one wishes to visualize a PCR fragment on an electrophoretic gel, a smaller fragment would suffice. However, for optimum PCR amplification, a fragment of 100 nucleotides is still preferred. Preferably in both cases, the fragment should exceed 150 nucleotides.

The primer should be chosen so that the two primers are not complementary at the 3′ ends. This situation would lead to a hybridization reaction between the primers before the primers hybridize to the substrate material. A complementary region of equal to or greater than 2 nucleotides will cause an unwanted primer hybridization. Preferably, there will be no complementary region at the 3′ end. Also preferred are primers that do not have internal complementary segments that allow formation of hairpins.

### B. AMPLIFICATION METHODS AND DETECTION OF PRODUCTS

The primers and oligonucleotide probes of the invention are used in methods of detecting the presence of (per)chlorate reducing bacteria. The presence of such bacteria is detected by determining the presence of the cld gene to which the degenerate primers of the present invention hybridize. The detection of the hybridized nucleotides is effected through the use of a polymerization chain reaction, a technique that is widely known in the field (U.S. Pat. Nos. 4,683,195; 4,683,202; and 4,800,159).

Nucleic acid amplification by template-directed, enzyme-dependent extension of primers is well known in the art. For example, amplification by the polymerase chain reaction (PCR) has been described. Details regarding various PCR methods, including, e.g., asymmetric PCR, reverse transcription-PCR, in situ PCR, quantitative PCR, real time PCR, and multiplex PCR, are well described in the literature. Details regarding PCR methods and applications thereof are found, e.g., in Sambrook et al., Molecular Cloning—A Laboratory Manual (3rd Ed.), Vol. 1-3, Cold Spring Harbor Laboratory, Cold Spring Harbor, N.Y. (2000); F. M. Ausubel et al. (eds.), Current Protocols in Molecular Biology, Current Protocols, a joint venture between Greene Publishing Associates, Inc. and John Wiley & Sons, Inc., (supplemented through 2002); Innis et al. (eds.), PCR Protocols: A Guide to Methods and Applications, Academic Press Inc., San Diego, Calif. (1990); J. P. V. Heuvel, PCR Protocols in Molecular Toxicology, CRC Press (1997); H. G. and A. Griffin, PCR Technology: Current Innovations, CRC Press (1994); Bagasra et al., (1997) In Situ PCR Techniques, Jossey-Bass; Bustin (2000) “Absolute quantification of mRNA using real-time reverse transcription polymerase chain reaction assays” Journal of Molecular Endocrinology 25:169-193; Poddar (2000) “Symmetric vs. asymmetric PCR and molecular beacon probe in the detection of a target gene of adenovirus” Molecular and Cellular Probes 14: 25-32; and Mackay et al. (2002) “Real-time PCR in virology” Nucleic Acids Res. 30:1292-1305, and references therein, among many other references.

Additional details regarding PCR methods, including asymmetric PCR methods, are found in the patent literature, e.g., U.S. Pat. No. 6,391,544 (May 21, 2002) to Salituro et al. entitled “Method for using unequal primer concentrations for generating nucleic acid amplification products”; U.S. Pat. No. 5,066,584 (Nov. 19, 1991) to Gyllensten et al. entitled “Methods for generating single stranded DNA by the polymerase chain reaction”; U.S. Pat. No. 5,691,146 (Nov. 25, 1997) to Mayrand entitled “Methods for combined PCR amplification and hybridization probing using doubly labeled fluorescent probes”; and U.S. patent application Ser. No. 10/281,054 (filed Oct. 24, 2002) by Beckman et al. entitled “Asymmetric PCR with nuclease-free polymerase or nuclease-resistant molecular beacons.”

The PCR reaction is achieved by repeated cycles of denaturation, annealing for hybridizing a target sequence of a sample with a complementary primer, and polymerization using a thermally stable DNA polymerase to extend a DNA double helix from the hybridized primer. If no nucleotide primer hybridizes to the target nucleic acid, there is no PCR product. The PCR primer acts as a hybridization probe.

In brief, PCR typically uses at least one pair of primers (typically synthetic oligonucleotides). Each primer hybridizes to a strand of a double-stranded nucleic acid target that is amplified (the original template may be either single-stranded or double-stranded). A pair of primers typically flanks a nucleic acid target that is amplified. Template-dependent extension of the primers is catalyzed by a DNA polymerase, in the presence of deoxyribonucleoside triphosphates (typically dATP, dCTP, dGTP, and dTTP, although these can be replaced and/or supplemented with other dNTPs, e.g., a dNTP comprising a base analog that Watson-Crick base pairs like one of the conventional bases, e.g., uracil, inosine, or 7-deazaguanine), an aqueous buffer, and appropriate salts and metal cations (e.g., Mg²⁺). The PCR process typically involves cycles of three steps: denaturation (e.g., of double-stranded template and/or extension product), annealing (e.g., of one or more primers to template), and extension (e.g., of one or more primers to form double-stranded extension products). The PCR process can instead, e.g., involve cycles of two steps: denaturation (e.g., of double-stranded template and/or extension product) and annealing/extension (e.g., of one or more primers to template and of one or more primers to form double-stranded extension products). The cycles are typically thermal cycles; for example, cycles of denaturation at temperatures greater than about 90° C., annealing at 50-75° C., and extension at 60-78° C. A thermostable enzyme is thus preferred.

Other suitable hybridization conditions for the PCR reaction will be well known to those of skill in the art. In certain applications, it is appreciated that lower stringency conditions may be required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Those of skill in the art will understand the salt concentrations and temperature parameters can be varied without departing from the nature of the invention.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C. Formamide and SDS also may be used to alter the hybridization conditions.

In specific preferred embodiments the annealing temperatures may range from 42° to 55° C., the MgCl₂ concentrations may be varied ranging from 1.0 to 3.0 mM, the primer amounts ranging from 15 to 60 pmol. In addition, the PCR methods were carried out in the presence of PCR additives, such as 0.25 mg of bovine serum albumin (BSA)/ml, 5% (vol/vol) dimethyl sulfoxide, and 1 M betaine. In the exemplary reaction mixtures employed in the present application, the reaction mixtures contained 1×Mg-free buffer, 1.0 to 3.0 mM MgCl₂, 200 μM of (each) deoxynucleoside triphosphates, 2.5 U of Taq polymerase (Sigma, St. Louis, Mo.), 1 μl of gDNA or environmental DNA, and nuclease-free double-distilled H₂O to a final volume of 50 μl. All components were purchased from Promega (Madison, Wis.) except for the polymerase.

In a particularly preferred PCR reaction, the following PCR conditions produced amplicons of the desired size using the DCD-F/DCD-R primer set: 44° C. annealing temperature, 60 pmol (each) primer, 1.5 mM MgCl₂, and 0.25 mg of BSA/ml. In the case of the UCD-238F/UCD-646R primer set, the optimal PCR conditions were 50° C. annealing temperature, 40 pmol (each) primer, 1.5 mM MgCl₂, and 0.25 mg of BSA/ml. Of course, the skilled artisan could vary these conditions and still achieve appropriate results in connection with the detection techniques of the present invention.

In the exemplary embodiments, the cycling parameters were as follows: reactions were initially heated to 94° C. for 2 min, followed by 30 cycles of 94° C. (1 min), annealing temperature (1 min), 72° C. (1 min), with a final 10-min 72° C. extension period. For touchdown cycling, the parameters consisted of a denaturation step at 94° C. for 1 min, a primer-annealing step for 1 min, and an extension step at 72° C. for 1 min. After 38 cycles, a final 10-min incubation was performed at 72° C. During the first 18 cycles, the annealing temperature was decreased by 1.0° C. every two cycles, starting at 59° C., until reaching a touchdown temperature of 50° C.

Automated thermal cyclers, including integrated systems for real time detection of product, are commercially available for performing PCR and other amplification reactions, e.g., the ABI Prism® 7700 sequence detection system from Applied Biosystems, the iCycler iQ® real-time PCR detection system from Bio-Rad, or the DNA Engine Opticon® continuous fluorescence detection system from MJ Research, Inc. In particularly preferred embodiments, the PCR reactions were performed in a Perkin-Elmer 2400 thermocycler (Applied Biosystems, Foster City, Calif.).

Thermostable enzymes (including Thermus aquaticus Taq DNA polymerase, as well as enzymes substantially lacking 5′ to 3′ nuclease activity), appropriate buffers, etc. are also widely commercially available, e.g., from Clontech (Palo Alto, Calif., USA), Invitrogen (Carlsbad, Calif., USA), Sigma-Aldrich (St. Louis, Mo., USA), and New England Biolabs (Beverley Ma, USA). For example, thermostable polymerases lacking 5′ to 3′ nuclease activity are commercially available, e.g., Titanium™ Taq (Clontech, Palo Alto, Calif., USA, www.clontech.com), KlenTaq DNA polymerase (Sigma-Aldrich, St. Louis, Mo., USA, www.sigma-aldrich.com), Vent™ and DeepVent™ DNA polymerase (New England Biolabs, Beverley Ma, USA, www.neb.com), Tgo DNA polymerase and FastStart DNA polymerase (Roche, Indianapolis, Ind., USA www.roche-applied-science.com), ABgene's Thermoprime Plus DNA Polymerase (Rochester, N.Y., USA), SuperTaq or SuperTaq Plus™ (Ambion, Austin, Tex., USA), FideliTaq™ DNA Polymerase (USB Corp., Cleveland, Ohio, USA, www.usb.com), Tfl DNA Polymerase (Promega, Madison, Wis., www.promega.com), and PfuTurbo® Cx Hotstart DNA Polymerase (Stratagene, La Jolla, Calif., USA, www.stratagene.com).

While it is preferred that the amplification reaction is the PCR reaction, there are other suitable amplification techniques such as CPR (Cycling Probe Reaction), bDNA (Branched DNA Amplification), SSR (Self-Sustained Sequence Replication), SOA (Strand Displacement Amplification), QBR (Q-Beta Replicase), Re-AMP (Formerly RAMP), NASBA (Nucleic Acid Sequence Based Amplification), RCR (Repair Chain Reaction), LCR (Ligase Chain Reaction), TAS (Transorbtion Based Amplification System), and HCS (amplifies ribosomal RNA), all of which may be used as the amplification method.

In one embodiment of the invention, this assay comprises the steps of exposing a nucleic acid isolated from a biological sample to oligonucleotide primers chosen from the group consisting of SEQ ID NO:1; SEQ ID NO:2; SEQ ID NO:3; SEQ ID NO:4; SEQ ID NO:5; SEQ ID NO:6, and SEQ ID NO:8, SEQ ID NO:9, SEQ ID NO:10 and SEQ ID NO:11. In specific embodiments, the PCR or transcription reaction is conducted directly on a bacterial lysate without isolating the nucleic acid therefrom. In other embodiments, nucleic acid is isolated from the sample, RNA is prepared therefrom and cDNA created from RNA. The reaction is allowed to incubate using PCR or other amplification or transcription conditions and the sample may then be examined for the presence of an amplification or other reaction product. In PCR, the method begins with exposing a biological sample to a primer pair specific for cld gene. More particularly, nucleic acid is isolated from the biological sample and contacted with the primer pairs. In certain embodiments, RNA is prepared from the nucleic acids. RNA is preferably isolated from biological samples by adding a guanidinium solution. Other methods known in the art of isolating RNA would also be suitable.

To perform the method of the present invention, one must first select a probe and primer pair of the present invention and expose the cld cDNA to the primer pair. After amplification, the PCR product is detected. In certain embodiments, performing PCR with SEQ ID NO:1 and 2 alone is sufficient. In other preferred embodiments, however, it is contemplated that a nested PCR is performed in which the amplification product from the PCR reaction with SEQ ID NO:1 and 2 (or alternatively, SEQ ID NO:8 and 9) is used as a template for a second PCR reaction in which the probes are primers SEQ ID NO:3 and 4. In alternative embodiments, the detection is carried out by using SEQ ID NO:5, 6, 10 or 11, instead of SEQ ID NO:1 2, 8 or 9, respectively.

In specific embodiments, it is contemplated that a reverse transcription reaction is carried out to determine the presence of cld mRNA in the sample. The reverse transcription reaction performed alone would be sufficient to identify the presence of the DPRB in the biological sample being tested. However, in other specific embodiments, it is contemplated that the reaction product from the reverse transcription reaction is employed as a template for a further PCR reaction in which a pair of primers described herein are used. Thus, in certain embodiments, it is contemplated that a method is carried out in which a reverse transcription reaction is carried out using a primer selected from the group consisting of SEQ ID NO:1 through SEQ ID NO:6. In yet another embodiment, a contemplated method involves performing such a reverse transcription reaction, isolating the product of the reaction and using it as a template for a PCR reaction using a pair of primers selected from the group consisting of SEQ ID NO:1 through SEQ ID NO:6. In an exemplary such embodiment, the reverse transcription reaction product is used as a template for a PCR reaction using the primer pair of SEQ ID NO:1/SEQ ID NO:2. In another exemplary embodiment, the reverse transcription reaction product is used as a template for a PCR reaction using the primer pair of SEQ ID NO:3/SEQ ID NO:4. In still another exemplary embodiment, the reverse transcription reaction product is used as a template for a PCR reaction using the primer pair of SEQ ID NO:5/SEQ ID NO:6. In additional embodiments, the reaction may be such that the reverse transcription reaction is followed by a first PCR reaction e.g., using a primer pair of SEQ ID NO:1/SEQ ID NO:2 or a primer pair of SEQ ID NO:5/SEQ ID NO:6, followed by a second PCR reaction using the less degenerate primer pair of SEQ ID NO:3/SEQ ID NO:4.

In performing the reverse transcription, once RNA is isolated from the biological sample, and exposed to reverse transcriptase enzyme and deoxyribonucleotides so that a cDNA molecule may be created that corresponds to the initial RNA molecule. Exemplary reverse transcriptases that may be used include, but are not limited to AMV Reverse Transcriptase (GEHealthcare and Amersham Biosciences, Piscataway, N.J., USA, AMV Reverse Transcriptase (Stratagene, La Jolla, Calif., USA www.stratagene.com), AMV RT (CHIMERx, Madison, Wis. USA, www.chimerx.com), cloned AMV Reverse Transcriptase (Invitrogen, Carlsbad, Calif., USA www.invitrogen.com), AMV Reverse Transcriptase (Ambion, Austin Tx, USA, www.ambion.com), AMV Reverse Transcriptase (www.MJResearch.com), AMV Reverse Transcriptase (Promega, Madison Wis., USA www.promega.com), and Reverse Transcriptase AMV (Roche, Indianapolis, Ind., USA, www.roche-applied-science.com)

For a PCR reaction, one would choose to use a pair of primers and examine the final product for presence of a PCR amplification product. This examination could involve examining the products of the reaction on an electrophoretic gel and determining whether an amplified product of the appropriate size had been created. One of skill in the art of molecular biology will be aware of many protocols designed to optimize PCR reactions. Particularly useful protocols are described in PCR Protocols, Ed. M. Innis, et al. Academic Press, San Diego.

The PCR reaction can be coupled to, for example, an ELISA detection procedure. In such a procedure, one would anchor the PCR amplification product to a solid support and examine the support for the presence of the PCR product. This procedure could be done in several ways. For example, one first attaches the amplified product to a solid support, such as a microtiter dish. For example, a streptavidin-coated plate may be provided. One of the selected primers may be attached to a biotin molecule so that an amplification product will be labelled with biotin and bind to the streptavidin plate. This is yet a further use of the probes of the present invention in that the primers/probes of the invention can be used to “fish-out” and identify/isolate the amplified product. For such embodiments, hybridization conditions discussed above could be used. In these embodiments, the plate and product are then exposed to cld-specific oligonucleotide probe of the invention containing a segment of the cld sequence. This probe is attached to a marker enzyme, such as horseradish peroxidase (HRP), which may be detected via its enzymatic properties.

In another method, one would attach a protein molecule capable of binding to the solid support, e.g., BSA, to an oligonucleotide probe/primer of the present invention. The plate is then coated with these protein-attached oligonucleotides, and these oligonucleotides are available to hybridize with an amplified product. This amplified product is preferably attached to a label molecule, such as biotin, that is capable of being detected. In one embodiment, the biotin-labelled PCR product may be complexed to a streptavidin-horse radish peroxidase conjugate. One may then detect this complex with the appropriate substrate.

The amplified products can be identified using various techniques, for example, by inserting a labeled nucleotide into the strands amplified using labeled primers. Examples of standard labeling materials include, but are not limited to, radioactive materials (³²P, ³⁵S, ¹³¹I, ¹²⁵I, ¹⁴C, ³H), fluorescent labels (e.g., fluorescein, Texas Red, rhodamine, BODIPY, resorufin or arylsulfonate cyanines, digoxygenin, horseradish peroxidase, alkaline phosphatases, acridium ester), chemiluminescent labels (e.g., acridinium esters), biotin, and jack beam urease. Furthermore, the PCR products obtained using non-labeled primers can be identified by combination of gel separation using electrophoresis and a dye-based visualizing technique. Thus it is particularly contemplated that the primers of the invention are labeled with such detectable labels.

A label or quencher can be incorporated into the primers and probes of the invention during oligonucleotide synthesis by using a specialized phosphoramidite including the label or quencher, or a modified base phosphoramidite including an alkyl spacer can be incorporated during oligonucleotide synthesis and the label or quencher can be linked to the spacer after synthesis is complete. As a specific example, fluorescein can be incorporated at the 5′ end of a probe of the invention by using a fluorescein phosphoramidite in the last step of the synthesis. As another specific example, a modified T including a C6 spacer with a primary amino group can be incorporated into the oligonucleotide, and a succinimidyl ester of 4-(4′-dimethylaminophenylazo)benzoic acid (DABCYL) can be attached to the primary amino group. (Such modified phosphoramidites are commercially available, e.g., AminoModifier C6 dT from Glen Research.) Similarly, 4-dimethylaminophenylazophenyl-4′-maleimide (DABMI) can be used when the site of attachment is a sulphydryl group.

As other examples, fluorescein can be introduced into oligonucleotides, either by using a fluorescein phosphoramidite that replaces a nucleoside with fluorescein, or by using a fluorescein dT phosphoramidite that introduces a fluorescein moiety at a thymidine ring via a spacer. To link a fluorescein moiety to a terminal location, iodoacetoamidofluorescein can be coupled to a sulphydryl group. Tetrachlorofluorescein (TET) can be introduced during automated synthesis using a 5′-tetrachloro-fluorescein phosphoramidite. Other reactive fluorophore derivatives and their respective sites of attachment include the succinimidyl ester of 5-carboxyrhodamine-6G (RHD) coupled to an amino group; an iodoacetamide of tetramethylrhodamine coupled to a sulphydryl group; an isothiocyanate of tetramethylrhodamine coupled to an amino group; or a sulfonylchloride of Texas red coupled to a sulphydryl group.

### C. ASSAYS AND KITS

In one embodiment, the present invention is an assay for the presence of DPRB in a sample. In other embodiments, this assay may be combined in an assay for at least one other microorganism that can effect remediation. For example, in contaminated soils or water samples, it may be desirable to remove the (per)chlorate contamination using DPRB and to remove contamination of other contaminants using other reclamation and/or bioremediation techniques. Preferably, such assays examine a biological sample for presence or absence of the appropriate contaminants as well as microorganisms that can remove such contaminants.

The cld gene may be derived from any DPRB. The presence of this gene in a sample being tested is indicative of the sample possessing bacteria that will effect remediation of the sample and clean-up any (per)chlorate contamination thereof. The organisms from which the cld gene may be detected and whose presence is desired in the samples to effect bioremediation of a (per)chlorate contaminated sample include, but are not limited to, Dechloromonas spp., Azoarcus spp., Dechlorospirillum spp., Dechloromarinus spp., Ideonella spp., Magnetospirillum spp., Pseudomonas spp., Rhodocyclus spp., Rhodospirillum spp., Azospirillum spp., Wolinella spp., and Xanthomonas spp. Exemplary bacteria from these species include for example, Dechloromonas agitata, Dechloromonas aromatica, Azospira suillum, Dechlorospirillum anomalous, Dechloromarinus chlorophilus, and Ideonella dechloratans. These are merely exemplary DPRBs and many others will be known to those of skill in the art.

In another embodiment, the present invention provides a kit for assaying DPRB present in a sample. In a preferred embodiment, the kit comprises a pair of primers selected from SEQ ID NOs:1-6. In another embodiment, the kit comprises at least one additional pair of primers designed to amplify a 16S RNA from one or more of the organisms indicated above. In a more preferred embodiment of the kit, the kit additionally comprises primers SEQ ID NO:1 and SEQ ID NO:2; in another embodiment, the kit comprises primers SEQ ID NO:3 and SEQ ID NO:4, is still a further embodiment, the kit comprises primers of SEQ ID NO:5 and SEQ ID NO:6. Kits that comprise SEQ ID NO:1, SEQ ID NO:2; SEQ ID NO:3 and SEQ ID NO:4 are specifically contemplated. Kits that comprise primers of the sequence SEQ ID NO:3, SEQ ID NO:4, SEQ ID NO:5 and SEQ ID NO:6 are specifically contemplated. Preferably, the primers of each SEQ ID NO are provided in separate containers.

In addition, the kits may comprise one or more enzymes for the PCR amplification and/or for the reverse transcription reactions. Thus, the kits optionally also includes one or more of: a polymerase (e.g., a polymerase having or substantially lacking 5′ to 3′ nuclease activity), a buffer, a standard template for calibrating a detection reaction, instructions for extending the primers to amplify at least a portion of the target nucleic acid sequence or reverse complement thereof, instructions for using the components to amplify, detect and/or quantitate the target nucleotide sequence or reverse complement thereof, or packaging materials. The kits may also preferably include the deoxyribonucleoside triphosphates (typically dATP, dCTP, dGTP, and dTTP, although these can be replaced and/or supplemented with other dNTPs, e.g., a dNTP comprising a base analog that Watson-Crick base pairs like one of the conventional bases, e.g., uracil, inosine, or 7-deazaguanine), an aqueous buffer, and appropriate salts and metal cations (e.g., Mg²⁺)

The kit may comprise a solid support on which the present the primers. Alternatively, the primers may be bound to a solid support. The solid support may be any support that is typically used to in nucleic acid preparation and analysis. Such supports include, but are not limited to plastic, glass, beads, microtiter plates. Indeed, glass, plastics, metals and the like are often used, and the nucleic acid amplification method of the present invention can be used irrespective of the type of the substrate. In some aspects, the probes of the invention may be effectively used for assaying nucleic acid molecules using a DNA chip or DNA micro-array where a large number of DNA probes are immobilized on a flat substrate. Furthermore, other than the flat DNA chips, the nucleic acid assay method using beads on which DNA probes are immobilized has become popular in recent years, and the nucleic acid amplification method of the present invention is also applicable to preparation of a sample in the nucleic acid detection method using the DNA probes immobilized on the surfaces of beads.

The primers in the kits may be provided in the form of nucleic acid-based arrays. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays. The term “microarray” refers to an ordered arrangement of hybridizable array elements. The array elements are arranged so that there are preferably at least two or more different array elements, more preferably at least 20 array elements, and more preferably at least 100 array elements, on a 1 cm² substrate surface. The hybridization signal from each of the array elements is individually distinguishable a specific location, or address of the probe. In a preferred embodiment, the array elements comprise polynucleotide primers of the present invention.

In addition to the above, the kits may comprises components as standards. For example, the kits may comprise a known cld sequences such that the signal received from the environmental/biological sample can be compared with that received from the standard to ensure the integrity of the assay components and conditions.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Bacterial strains, environmental samples, and DNA extraction. The bacterial strains and environmental samples used in the present application are listed in Table 1. Genomic DNA from pure cultures was extracted by using the PUREGENE DNA isolation kit (Gentra Systems Inc., Minneapolis, Minn.), DNA was extracted from environmental samples by using the Fast DNA Spin kit for soil (Qbiogene, Carlsbad, Calif.). DNA for PCR from the Los Alamos well most probable-number samples was obtained by harvesting the cell pellet from 1.5 ml of culture, adding 40 μl of sterile H₂0 and 5 μl of chloroform, and lysing the cells by heating them at 95° C. for 10 min.

TABLE 1
Bacterial strains and environmental samples used in this study

| | |
|---|---|
| Origin of DNAs tested | Description |

| | |
|---|---|
| Strains | |
| Dechloromonas agitara | (Per)chlorate reducer |
| “Dechloromonas aromatica” | (Per)chlorate reducer |
| Dechloromonas sp. strain I.T1 | (Per)chlorate reducer |
| Dechlorosoma suillian | (Per)chlorate reducer |
| “Dechloromarinus chlorophilus” strain | Chlorate reducer |
| NSS | |
| “Dechlorospirillum anomalous“ strain | (Per)chlorate reducer |
| WD | |
| Pseudomonas sp. strain PK | Chlorate reducer |
| Strain CR | (Per)chlorate reducer |
| Dechlorospirillum sp. strain DB | (Per)chlorate reducer |
| Ideonella dechloratans | Chlorate reducer |
| Pseudomonas stutzeri | Non-perchlorate reducer |
| Magnetospirillum magnetotacticum | Non-perchlorate reducer |
| Rhodocyclus renuis | Non-perchlorate reducer |
| Escherichia coli | Non-perchlorate reducer |
| Environmental samples | |
| Vida | Antarctica, diesel- |
| | contaminated site |
| Lake Fryxell sediment | Antarctica, pristine site |
| Lake Fryxell water column | Antarctica, pristine site |
| Lake Hoare mat | Antarctica, pristine site |
| Lake Hoare water column | Antarctica, pristine site |
| campus lake | Southern Illinois University |
| library pond | Southern Illinois University |
| library soil | Southern Illinois University |
| Los Alamos wells | MPNs from perchlorate- |
| | contaminated site in Los |
| | Alamos, N.M.^{a} |

^{a}MPNs, most-probable-number samples.

PCR primers and reaction conditions. Primer sets targeting the chlorite dismutase gene were designed based on areas of amino acid and nucleotide sequence conservation. These areas of conservation were visualized by manual sequence alignment using the Se—Al (Rambaut, Sequence Alignment Editor v.2.0, Dept. of Zoology, University of Oxford, Oxford, UK, 2000) program. The primers DCD-F [5′-GA (A/G)CGCAA(A/G)(A/G)GNGCNGCNG(A/C)NGA(A/G)GT-3′] (SEQ ID NO:1) and DCD-R [5′-TC(A/G)AA(A/G)TANGT(A/T/G)AT(A/G)AA(A/G)TC-3′] (SEQ ID NO:2) were developed based on the amino acid conservation of the cld sequences from Dechloromonas agitata, Dechloromonas aromatica, Ideonella dechloratans, and M. magnetotacticum. The primers UCD-238F [5′-T(C/T)GA(A/C/G)AA(A/G)CA(C/T)AAGGA(A/T/C)AA(A/C/G)GT-3′] (SEQ ID NO:3) and UCD-646R [5′-GAGTGGTA(A/C/G)A(A/G)(C/T)TT(A/C/G)CG(C/T)TT-3′] (SEQ ID NO:4) were developed from an expanded alignment that also included the cld gene sequences from Pseudomonas sp. strain PK, Dechloromonas sp. strain LT1, Azospira suillum, “Dechlorospirillum anomalous” strain WD, and “Dechloromarinus chlorophilus” sp. strain NSS. Primers were synthesized by Integrated DNA Technologies, Coralville, Iowa.

To optimize PCR conditions, annealing temperatures ranging from 42° to 55° C., MgCl₂ concentrations ranging from 1.0 to 3.0 mM, primer amounts ranging from 15 to 60 pmol, and PCR additives, such as 0.25 mg of bovine serum albumin (BSA)/ml, 5% (vol/vol) dimethyl sulfoxide, and 1 M betaine, were tested. PCRs were performed in a Perkin-Elmer 2400 thermocycler (Applied Biosystems, Foster City, Calif.).

All reaction mixtures consisted of 1×Mg-free buffer, 1.0 to 3.0 mM MgCl₂, 200 μM (each) deoxynucleoside triphosphates, 2.5 U of Taq polymerase (Sigma, St. Louis, Mo.), 1 μl of gDNA or environmental DNA, and nuclease-free double-distilled H₂0 to a final volume of 50 μl. All components were purchased from Promega (Madison, Wis.) except for the polymerase.

The following PCR conditions produced amplicons of the desired size using the DCD-F/DCD-R primer set: 44° C. annealing temperature, 60 pmol (each) primer, 1.5 mM MgCl₂, and 0.25 mg of BSA/ml. Optimal PCR conditions for the UCD-238F/UCD-646R primer set were 50° C. annealing temperature, 40 pmol (each) primer, 1.5 mM MgCl₂, and 0.25 mg of BSA/ml.

Normal cycling parameters were as follows: reactions were initially heated to 94° C. for 2 min, followed by 30 cycles of 94° C. (1 min), annealing temperature (1 min), 72° C. (1 min), with a final 10-min 72° C. extension period. For touchdown cycling, the parameters consisted of a denaturation step at 94° C. for 1 min, a primer-annealing step for 1 min, and an extension step at 72° C. for 1 min. After 38 cycles, a final 10-min incubation was performed at 72° C. During the first 18 cycles, the annealing temperature was decreased by 1.0° C. every two cycles, starting at 59° C., until reaching a touchdown temperature of 50° C.

To verify the integrity of the amplification, both positive and negative (no template DNA) reactions were included. PCR results were checked using agarose gel electrophoresis on a 2% agarose gel containing 1×Tris-acetate-EDTA buffer.

Cloning and sequencing of PCR products. PCR products of the appropriate size were gel extracted and subjected to the Geneclean Spin kit (Qbiogene) for subsequent analysis. Products from gel purification were directly cloned into the pCR 2.1 TOPO vector (Invitrogen, Carlsbad, Calif.). The inserts were sequenced with vector primers, using a ThermoSequenase cycle sequencing kit (U.S. Biochemicals, Cleveland, Ohio) and [α-³⁵S]dATP as the label. Sequencing reactions were analyzed by electrophoresis through a 6% polyacrylamide-bisacrylamide gel.

Chlorite dismutase nucleotide sequences were manually entered using the MacVector 7.0 computer program (Oxford Molecular Group) and then transferred to Se—Al (Rambaut, Sequence Alignment Editor v.2.0, Dept. of Zoology, University of Oxford, Oxford, UK, 2000) for alignment. For 16S rDNA analysis, gene sequences were obtained from GenBank and 1,424 bases were aligned in the Seq-App computer program (Gilbert, SeqApp 1.9a157 ed. Biocomputing Office, Biology Dept., Indiana University, Bloomington, Indiana, USA).

Phylogenetic trees based on these alignments were constructed by using the PAUP* software package (beta version 4.0) (Swofford et al., “Phylogenetic Analysis Using Parsimony (an other methods) Version 4.0 Sinauer Associates, Inc. Sunderland, Mass., pp 407-514, 1999). Unrooted trees for the chlorite dismutase gene and the 16S rDNA gene were constructed by using the absolute-number-of-differences parameter within the distance criterion. This parameter was chosen based on the closely related protein coding sequence of the cld gene (Swofford et al., “Phylogenetic Inference” in D. M. Hollis et al., (Eds.) Molecular Systematics, 2^{nd} ed. Sinauer Associates, Inc. Sunderland, Mass., pp 407-514, 1996). However, separate analyses using the Kimura 2 parameter to correct for evolutionary distances as well protein alignments were also performed for comparison. Gaps were removed from the 16S rDNA data set. Trees were drawn using neighbor joining, and 100 replicates were performed in bootstrap analysis.

GenBank sequence accession numbers. Chlorite dismutase sequences generated from this study have been deposited in the GenBank database under the accession numbers AY540957 to AY540971. Chlorite dismutase gene sequences from the following strains were used in primer development: D. agitata (accession number AY124796), I. dechloratans (AJ296007), and M. magnetotacticum (ZP_{—}00053098). 16S rDNA sequences from the following strains were used for phylogenetic tree construction: D. agitata (AF047462), “D. aromatica” (AY032610), D. suillum (AF170348), “D. anomalous” strain WD (AF170352), “D. chlorophilus” sp. strain NSS (AF170359), Pseudomonas sp. strain PK (AF170358), Dechloromonas sp. strain LT1 (AY124797), strain CR (AY530552), Dechlorospirillum sp. strain DB (AY530551), I. dechloratans (X72724), and M. magnetotacticum (Y10110).

### Example 2

### Results and Discussion of Primer Design

In order to develop universal primers targeting the cld gene, complete gene sequences were aligned from D. agitata, “D. aromatica” (identified from the complete genome sequence provided by the Department of Energy Joint Genome Institute), I. dechloratans, and M. magnetotacticum (identified by cld hybridization and analysis of the complete genome) (Bender et al., Appl. Environ. Microbiol. 68:4820-4826, 2002). Visual alignment indicated sequence divergence at the 5′ end, while the 3′ end of the cld gene appeared more conserved (FIG. 1). This observation was expected based on previous hybridization analysis of several DPRB gDNAs using the D. agitata cld gene probe (Bender et al., Appl. Environ. Microbiol. 68:4820-4826, 2002).

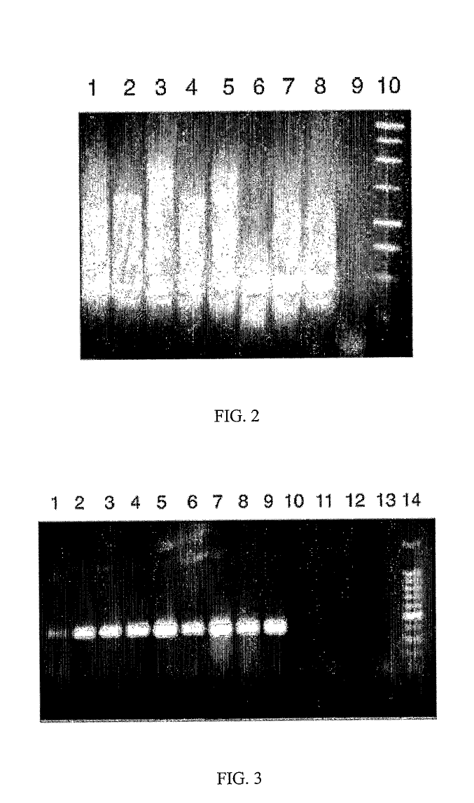

From the four aligned sequences, two areas of amino acid conservation were chosen, and PCR primers targeting all corresponding codons were developed (FIG. 1). Due to the limited alignment file, primer DCD-F (SEQ ID NO:1) contained 9 degenerate sites out of 27 nucleotide positions, while DCD-R (SEQ ID NO:2) contained 6 degenerate sites out of 20 nucleotide positions. This primer set was tested on five other DPRB for accuracy and ability to amplify the cld gene. While a band at 484 bp resulted with all DPRB tested, an abundance of spurious by-products were also observed (FIG. 2). When the gDNA template was diluted, no increase in specificity occurred with the DCD-F/DCD-R primer set; thus, the spurious by-products were most likely caused by the extreme degeneracy of the DCD-F/DCD-R primer set. Based on this lack of specificity of the DCD-F/DCD-R primer set, no negative control strains were tested using this primer set.

To increase the number of cld sequences represented in the alignment file and potentially reduce primer degeneracy, the 484-bp amplification product was excised from the gel, purified, cloned, and sequenced from DPRB Pseudomonas sp. strain PK, D. suillum, “D. chlorophilus,” “D. anomalous,” and Dechloromonas sp. strain LT1. These cld sequences, excluding priming sites, were added to the alignment file, and two areas of nucleotide conservation were targeted for primer design (FIG. 1).

Primer UCD-238F (SEQ ID NO:3) contained 6 degeneracies out of 22 bases, while primer UCD-646R (SEQ ID NO:4) contained 5 degeneracies out of 20 bases. A 408-bp product was visible in all DPRB gDNAs tested with little background amplification (FIG. 3). No amplification occurred in non-(per)chlorate-reducing strains, including Rhodocyclus tenuis and Pseudomonas stutzeri, both close phylogenetic relatives of DPRB strains but unable to reduce (per)chlorate. However, this primer set was unsuccessful in amplifying cld gene sequences from environmental samples known to contain DPRB. This result is explained by a lower concentration of target DNA in the environmental sample versus gDNA from pure cultures, as well as interference by nontarget DNA likely present in the environmental samples. Since specific products were obtained via PCR amplification using 16S rDNA primers on the environmental DNAs, it is unlikely that PCR inhibitors affected the detection process.

This problem was addressed by employing a nested PCR technique using the DCD-F/DCD-R primer set in an initial PCR, followed by a second amplification using the internal UCD-238F/UCD-646R primer set. Touchdown PCR cycling parameters were used to reduce the number of nontarget amplicons in the first PCR. Reaction products from the first amplification were diluted 1:10 and used as templates for the second round of amplification with the UCD-238F/UCD-646R primer set.

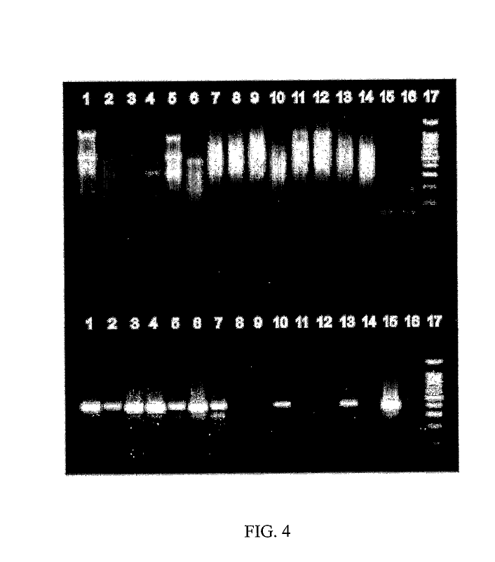

Results from the nested procedure indicated that this technique was successful in amplifying cld sequences from Pseudomonas sp. strain PK and Los Alamos well samples as positive controls and from certain experimental environmental samples (FIG. 4). While spurious reaction products were observed from most samples in the first round of amplification, a second-round product of 408 bp was clearly visible in several of the environmental samples, including the Southern Illinois University campus library pond, the pristine Lake Fryxell sediment and Lake Hoare 12-m water column, the diesel-contaminated Vida, and all five samples obtained from a perchlorate contaminated site in Los Alamos, N.M. (FIG. 4) known to contain DPRB. While no products of the appropriate size were evident in the first-round reaction for the Los Alamos-well 3 and Vida samples, an intense signal was present following the nested reaction. This result indicates that a low concentration of product was present in the first-round reaction, likely caused by a lower concentration of target DNA in these two samples. Thus, the nested procedure increases the sensitivity of this detection method.

### Example 3

### Results and Discussion of Sequence Analysis of the Products

Sequence analysis of the nested amplification products from Los Alamos-well 3, Los Alamos-well 4, Lake Fryxell sediment, Lake Hoare 12-m water column, and Vida samples indicated that all of the products were indeed cld gene sequences. While the Los Alamos well 3 clone was identical to the “D. aromatica” cld sequence, the Los Alamos well 4, Lake Fryxell sediment, Lake Hoare 12-m water column, and Vida clones were all most similar (amino acid similarity, 98.4 to 81.3%) to sequences from “D. anomalous” and strain DB (Table 2). The presence of cld sequences in the Antarctic samples was expected due to previously obtained DPRB isolates from these sites.

TABLE 2
Amino acid identities of partial chlorite dismutase protein sequences

| Source or organism | % Amino acid identity with sample no: | | | | | | | | | |
|---|---|---|---|---|---|---|---|---|---|---|
| and ID no.^{a} | 1 | 2 | 3 | 4 | 5 | 6 | 7 | 8 | 9 | 10 |
| 1. D. agitata | | 74.8 | 76.4 | 64.2 | 78.9 | 65.0 | 63.4 | 64.2 | 62.6 | 69.1 |
| 2. M. magneto | 74.8 | | 80.5 | 65.9 | 77.2 | 66.7 | 65.0 | 65.9 | 64.2 | 81.3 |
| 3. I. dechloratans | 76.4 | 80.5 | | 64.2 | 78.9 | 65.0 | 63.4 | 63.4 | 63.4 | 72.4 |
| 4. “D. aromatica” | 64.2 | 65.9 | 64.2 | | 64.2 | 98.4 | 98.4 | 97.6 | 95.9 | 66.7 |
| 5. Dcm. strain LT1 | 78.9 | 77.2 | 78.9 | 64.2 | | 65.0 | 63.4 | 64.2 | 62.6 | 73.2 |
| 6. Pseud. strain PK | 65.0 | 66.7 | 65.0 | 98.4 | 65.0 | | 98.4 | 95.9 | 95.1 | 68.3 |
| 7. “D. chlorophilus” | 63.4 | 65.0 | 63.4 | 98.4 | 63.4 | 98.4 | | 95.9 | 95.1 | 66.7 |
| 8. D. suillum | 64.2 | 65.9 | 63.4 | 97.6 | 64.2 | 95.9 | 95.9 | | 95.1 | 66.7 |
| 9. Strain CR | 62.6 | 64.2 | 63.4 | 95.9 | 62.6 | 95.1 | 95.1 | 95.1 | | 65.0 |
| 10. “D. anomalous” | 69.1 | 81.3 | 72.4 | 66.7 | 73.2 | 68.3 | 66.7 | 66.7 | 65.0 | |
| 11. Dsp. strain DB | 68.3 | 82.1 | 72.4 | 65.9 | 73.2 | 66.7 | 65.0 | 65.9 | 64.2 | 95.9 |
| 12. Los Alamos well 3 | 64.2 | 65.9 | 64.2 | 100 | 64.2 | 98.4 | 98.4 | 97.6 | 95.9 | 66.7 |
| 13. Los Alamos well 4 | 74.0 | 82.1 | 71.5 | 65.9 | 77.2 | 66.7 | 65.0 | 65.9 | 64.2 | 82.9 |
| 14. Lake Hoare 12m-A | 74.8 | 81.3 | 72.4 | 66.7 | 78.0 | 67.5 | 65.9 | 66.7 | 65.0 | 83.7 |
| 15. Lake Hoare 12m-B | 73.2 | 79.7 | 70.7 | 65.9 | 76.4 | 65.9 | 65.0 | 65.9 | 64.2 | 82.1 |
| 16. Lake Fryxell sed-A | 72.4 | 78.9 | 69.9 | 64.2 | 76.4 | 65.0 | 63.4 | 64.2 | 62.6 | 81.3 |
| 17. Lake Fryxell sed-B | 73.2 | 79.7 | 70.7 | 65.0 | 77.2 | 65.9 | 64.2 | 65.0 | 63.4 | 82.1 |
| 18. Vida-A | 69.1 | 82.9 | 72.4 | 66.7 | 73.2 | 68.3 | 66.7 | 66.7 | 65.0 | 97.6 |
| 19. Vida-B | 69.9 | 82.9 | 73.2 | 67.5 | 74.0 | 69.1 | 67.5 | 67.5 | 65.9 | 98.4 |

| Source or organism | % Amino acid identity with sample no: | | | | | | | | |
|---|---|---|---|---|---|---|---|---|---|
| and ID no.^{a} | 11 | 12 | 13 | 14 | 15 | 16 | 17 | 18 | 19 |
| 1. D. agitata | 68.3 | 64.2 | 74.0 | 74.8 | 73.2 | 72.4 | 73.2 | 69.1 | 69.9 |
| 2. M. magneto | 82.1 | 65.9 | 82.1 | 81.3 | 79.7 | 78.9 | 79.7 | 82.9 | 82.9 |
| 3. I. dechloratans | 72.4 | 64.2 | 71.5 | 72.4 | 70.7 | 69.9 | 70.7 | 72.4 | 73.2 |
| 4. “D. aromatica” | 65.9 | 100 | 65.9 | 66.7 | 65.9 | 64.2 | 65.0 | 66.7 | 67.5 |
| 5. Dcm. strain LT1 | 73.2 | 64.2 | 77.2 | 78.0 | 76.4 | 76.4 | 77.2 | 73.2 | 74.0 |
| 6. Pseud. strain PK | 66.7 | 98.4 | 66.7 | 67.5 | 65.9 | 65.0 | 65.9 | 68.3 | 69.1 |
| 7. “D. chlorophilus” | 65.0 | 98.4 | 65.0 | 65.9 | 65.0 | 63.4 | 64.2 | 66.7 | 67.5 |
| 8. D. suillum | 65.9 | 97.6 | 65.9 | 66.7 | 65.9 | 64.2 | 65.0 | 66.7 | 67.5 |
| 9. Strain CR | 64.2 | 95.9 | 64.2 | 65.0 | 64.2 | 62.6 | 63.4 | 65.0 | 65.9 |
| 10. “D. anomalous” | 95.9 | 66.7 | 82.9 | 83.7 | 82.1 | 81.3 | 82.1 | 97.6 | 98.4 |
| 11. Dsp. strain DB | | 65.9 | 82.9 | 83.7 | 82.1 | 81.3 | 82.1 | 96.7 | 97.6 |
| 12. Los Alamos well 3 | 65.9 | | 65.9 | 66.7 | 65.9 | 64.2 | 65.0 | 66.7 | 67.5 |
| 13. Los Alamos well 4 | 82.9 | 65.9 | | 99.2 | 97.6 | 96.7 | 96.7 | 83.7 | 83.7 |
| 14. Lake Hoare 12m-A | 83.7 | 66.7 | 99.2 | | 98.4 | 97.6 | 97.6 | 83.7 | 84.6 |
| 15. Lake Hoare 12m-B | 82.1 | 65.9 | 97.6 | 98.4 | | 95.9 | 95.9 | 82.1 | 82.9 |
| 16. Lake Fryxell sed-A | 81.3 | 64.2 | 96.7 | 97.6 | 95.9 | | 98.4 | 81.3 | 82.1 |
| 17. Lake Fryxell sed-B | 82.1 | 65.0 | 96.7 | 97.6 | 95.9 | 98.4 | | 82.1 | 82.9 |
| 18. Vida-A | 96.7 | 66.7 | 83.7 | 83.7 | 82.1 | 81.3 | 82.1 | | 99.2 |
| 19. Vida-B | 97.6 | 67.5 | 83.7 | 84.6 | 82.9 | 82.1 | 82.9 | 99.2 | |

^{a}ID no., identification no. for sample (numbers correspond to numbers in column heads); Dcm strain LT1. Dechloromonas sp. strain LT1; Pseud strain PK, Pseudomonas sp. strain PK; Dsp. strain DB, Dechlorospirillum sp. strain DB; Lake Hoare 12m-A and 12m-B, clones A and B from 12-m water column of Lake Hoare; Lake Fryxell sed-A and sed-B, clones A and B from Lake Fryxell sediment; Vida-A and Vida-B, clones A and B from Vida.

Sequence analysis indicated that more than one phylotype was present in samples collected from Vida, the Lake Hoare 12-m water column, and the Lake Fryxell sediment. Although these differences were only one or two nucleotides, the predicted protein products reflected these changes (Table 2). The observation of different cld gene sequences from the same environmental sample indicates the presence of more than one DPRB strain, and as such, denaturing gradient gel electrophoresis may be a useful tool in determining the number of and prevalent phylotypes in a given sample (Karr et al., Appl. Environ. Microbiol., 69:4910-4914, 2003). Since denaturing gradient gel electrophoresis could also be used to address the effect of ecological changes on the diversity of cld sequences present, the nested cld primer sets could be used to analyze and monitor DPRB populations in the environment.

Aside from the biases of PCR, this detection method is more inclusive than 16S rDNA primer sets, which can detect only a few genera of DPRB. However, a limitation of these primer sets is that only cld genes with sequences similar to those of the priming sites will be detected. This detection method would overlook extremely diverse sequences due to primer development from what is believed to be a minimum sampling of cld genes. Because the primer sets can detect cld genes in a DNA sample, the nested PCR approach does not require that the cells be actively reducing (per)chlorate and, as such, is useful for assessing the (per)chlorate-reducing potential of an environment. Although DNA:DNA hybridization studies have also be used to detect the cld gene (Bender et al., Appl. Environ. Microbiol. 68:4820-4826, 2002), this approach requires more target DNA than a PCR-based approach, and hybridization signals could be affected by interference from environmental constituents.

Because this detection method targets a single gene in the metabolic pathway, it is possible to obtain false positives, as evidenced by M. magnetotacticum, an organism that harbors the cld gene but lacks other genes, such as those for (per)chlorate reductase, required for perchlorate reduction. However, subsequent analyses of cld-positive environmental samples, using (per)chlorate reductase probes, should eliminate these false positives from further consideration. While the nested PCR approach is efficient at detecting cld genes in the environment, traditional PCR cannot be used to determine the relative abundance or activity of DPRB in a given site. Based on the lack of perchlorate in most environments and the ability of DPRB to use alternate metabolisms, there is some question that the organisms detected using these primers are actively reducing perchlorate. For these analyses, the cld primer sets could be used in quantitative and reverse transcription-PCR. These strategies could also be used to monitor the sustainability of natural attenuation over long periods of time. Smets and colleagues observed that biodegradation of chlorinated solvents decreased over a 2-month period due to physiological changes of the bacteria in response to the environment (Environ. Microbiol., 4:315-317, 2002). Thus, the cld primer sets could be used in an RNA approach to assess the long-term attenuation potential of a bacterial community. Quantitative PCR using this primer set could also determine if an increase in catabolic gene copy number occurs after a growth amendment is exogenously supplied. An increase in gene copy number would imply that the perchlorate reducing potential of the site had been enhanced and that stimulation of these bacteria may lead to the natural attenuation of perchlorate. Thus, quantitative PCR using a metabolic primer set could aid in monitoring the effectiveness of a bioremediative strategy.

### Example 4

### Results and Discussion of Phylogeny of cld Gene

From the development of the degenerate cld primer set, the first library of cld gene sequences was generated. Included in this library were cld sequences from strains DB and CR, two perchlorate-reducing strains isolated during the cld primer development. Both strains originated from perchlorate-contaminated sites in Los Alamos, N.M. From the 16S rDNA sequence, strain DB was designated a Dechlorospirillum species within the Rhodospirillaceae of the alpha-Proteobacteria, and strain CR was designated a member of the Rhodocyclus assemblage within the beta-Proteobacteria.

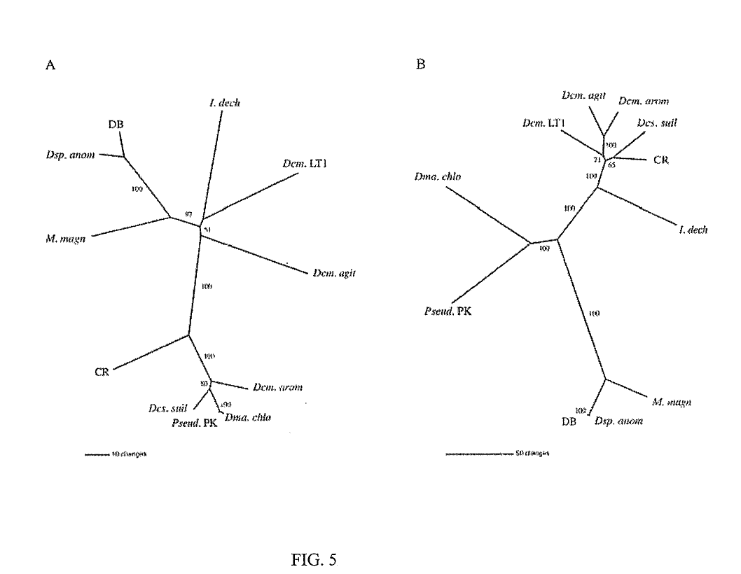

To determine if the c/d gene phylogeny tracked that of the 16S rDNA gene and to possibly gain some insight into the evolution of (per)chlorate reduction, unrooted phylogenetic trees were compared. Comparison of the cld and 16S rDNA gene trees resulted in incongruent topologies (FIG. 5). While M. magnetotacticum, “D. anomalous,” and Dechlorospirillum strain DB, all members of the alpha-Proteobacteria, form a distinct cluster on both trees, the cld gene sequences from “D. aromatica,” D. suillum, and strain CR (all members of the beta-Proteobacteria) cluster with those from the gamma-Proteobacteria Pseudomonas sp. strain PK and “D. chlorophilus.” This aberration indicates that although D. agitata, “D. aromatica,” and Dechloromonas sp. strain LT1 are all members of the same genus, their respective cld gene sequences are not monophyletic. In addition, extremely short branch lengths on the cld tree among D. suillum, “D. aromatica,” Pseudomonas sp. strain PK, and “D. chlorophilus” reflect the high sequence similarity of these genes and indicate possible transfer of the cld gene among these members of the beta- and gamma-Proteobacteria (FIG. 5). These incongruent tree topologies suggest a role for horizontal gene transfer in the evolution of the (per)chlorate reduction pathway. This conclusion is based on previous studies regarding incongruent tree topologies and the occurrence of gene conservation among diverse hosts as evidence of horizontal transfer (Herrick et al., Appl. Environ. Microbiol., 63:2330-2337, 1997; Klein et al., J. Bacteriol. 183:6028-6035, 2001, Koonin et al., Ann. Rev. Microbiol., 55:709-742, 2001). Preliminary G+C content analysis of “D. aromatica” and M. magnetotacticum genomes also implicates the involvement of horizontal transfer with the spread of the cld gene. The G+C content of the “D. aromatica” genome is 59.2% (http://genome.ornl.gov/microbial/daro/), while the C+C content of the cld gene is 49.7%. Similarly, the G+C content of the M magnetotacticum genome is 64.0% (http://genome.ornl.gov/microbial/mmag/), while the G+C content of the cld gene is 52.4%.

Due to the conserved nature of chlorite dismutase and the unambiguous nucleotide sequence alignment (FIG. 1), it is doubtful that the tree topology is incorrect. Trees constructed utilizing the Kimura 2 parameter and those constructed from amino acid alignments for the cld gene product resulted in similar topologies. The incongruent tree topologies could alternatively be explained by a series of gene duplication and deletion events. However, in this case, the resulting cld gene sequences would still be expected to be similar to those of close phylogenetic relatives. Thus, both the cld gene sequence diversity and metabolic diversity of DPRB may be a direct result of horizontal transfer. Since DPRB can grow by alternate metabolisms, the cld gene may not be subject to intense selective pressure. As such, mutation may occur until the gene sequence becomes functional with respect to the codon usage and regulation of the host. However, more extensive data are needed on the codon biases and G+C content of housekeeping genes in other DPRB isolates before further conclusions can be drawn. While one can only speculate on the possible mechanism of transfer, a transposase gene was identified directly upstream of the cld gene in Pseudomonas sp. strain PK. Other genes involved in the perchlorate reduction pathway were also identified in the direct proximity of the cld gene in D. agitata and “D. aromatica,” indicating that this metabolism may have been conferred through the action of a mobile genetic element. While phylogenetic comparisons of the cld gene and the 16S rDNA gene indicate that horizontal transfer is involved in the evolution of (per)chlorate metabolism, an interesting question still remains regarding the progenitor of (per)chlorate reduction and the selective advantage for retaining this metabolic machinery given that (per)chlorate has been widespread in the environment only in the last 50 years and that many DPRB are found in pristine areas.

The discussion in the present example reveals the significance of using DPRB in environmental bioremediation techniques. The previous examples have provided detailed and exemplary methods for identifying the DPRB in accordance with the present invention. It should be understood that the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. A composition comprising a first primer and a second primer, wherein the first primer has a nucleic acid sequence that comprises a sequence of SEQ ID NO:1 or SEQ ID NO:8 and the second primer has a nucleic acid sequence that comprises a sequence of SEQ ID NO:2 or SEQ ID NO:9, wherein said first and second primers are capable of hybridizing to a chlorite dismutase (cld) gene.

2. The composition of claim 1, further comprising a third primer and a fourth primer, wherein the third primer has a nucleic acid sequence that comprises a sequence of SEQ ID NO:3 and the fourth primer has a nucleic acid sequence that comprises a sequence of SEQ ID NO:4, wherein said third and fourth primers are capable of hybridizing to a cld gene.

3. The composition of claim 1, further comprising a third primer and a fourth primer, wherein the third primer has a nucleic acid sequence that comprises a sequence of SEQ ID NO:5 or SEQ ID NO:10 and the fourth primer has a nucleic acid sequence that comprises a sequence of SEQ ID NO:6 or SEQ ID NO:1, wherein said third and fourth primers are capable of hybridizing to a cld gene.

4. A composition comprising a first primer and a second primer, wherein the first primer has a nucleic acid sequence that comprises a sequence of SEQ ID NO:3 and the second primer has a nucleic acid sequence that comprises a sequence of SEQ ID NO:4, wherein said first and second primers are capable of hybridizing to a chlorite dismutase (cld) gene.

5. The composition of claim 4, further comprising a third primer and a fourth primer, wherein the third primer has a nucleic acid sequence that comprises a sequence of SEQ ID NO:5 or SEQ ID NO:10 and the fourth primer has a nucleic acid sequence that comprises a sequence of SEQ ID NO:6 or SEQ ID NO:1, wherein said third and fourth primers are capable of hybridizing to a cld gene.

6. The composition of claim 2, further comprising a fifth primer and a sixth primer, wherein the fifth primer has a nucleic acid sequence that comprises a sequence of SEQ ID NO:5 or SEQ ID NO:10 and the sixth primer has a nucleic acid sequence that comprises a sequence of SEQ ID NO:6 or SEQ ID NO:11, wherein said fifth and sixth primers are capable of hybridizing to a cld gene.

7. (canceled)

8. (canceled)

9. (canceled)

10. The composition of claim 1 wherein the cld gene is from dissimilatory (per)chlorate-reducing bacteria (DPRB) species.

11. The composition of claim 10, wherein said DPRB is a bacterium from the Dechloromonas spp., Azoarcus spp., Dechlorospirillum spp., Dechloromarinus spp., Ideonella spp., Magnetospirillum spp., Pseudomonas spp., Rhodocyclus spp., Rhodospirillum spp., Azospirillum spp., Wolinella spp., Xanthomonas spp.

12. The composition of claim 11, wherein said DPRB is selected from the group consisting of Dechloromonas agitate, Dechloromonas aromatica, Azospira suillum, Dechlorospirillum anomalous, Dechloromarinus chlorophilus, Ideonella dechloratans, and Magnetospirillum magnetotacticum.

13. (canceled)

14. An oligonucleotide primer pair wherein the first primer of the primer pair comprises a sequence of SEQ ID NO:1 or SEQ ID NO:8 and the second primer of the primer pair comprises a sequence of SEQ ID NO:2 or SEQ ID NO:9.

15. An oligonucleotide primer pair wherein the first primer of the primer pair comprises a sequence of SEQ ID NO:3 and the second primer of the primer pair comprises a sequence of SEQ ID NO:4.

16. (canceled)

17. (canceled)

18. An oligonucleotide primer which has the nucleotide sequence defined in any one of SEQ ID NOs: 1, 2, 3, 4, 5, 6, 8, 9, 10, or 11.

19. (canceled)

20. (canceled)

21. (canceled)

22. (canceled)

23. (canceled)

24. A method of detecting the presence of (per)chlorate reducing bacteria in a sample comprising:
(a) subjecting DNA of bacterial cells in said sample to a first polymerase chain reaction amplification using a pair of primers of claim 14; and
(b) detecting the product or products of said first polymerase chain reaction amplification, thereby identifying the presence of said (per)chlorate-reducing bacteria in said sample.

25. (canceled)

26. (canceled)

27. (canceled)

28. (canceled)

29. (canceled)

30. The method of, claim 24 wherein said sample is a water sample.

31. The method of, claim 24 wherein said sample is a soil sample.

32. The method of claim 30, wherein said water sample is collected from a water supply that has been contaminated with perchlorate.

33. The method of claim 31, wherein said soil sample is collected from land that has been contaminated with perchlorate.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method of determining whether a sample contains bacteria that is reducing (per)chlorate in said sample comprising:
(a) isolating nucleic acid from said sample;
(b) incubating said nucleic acid with a DNase to isolate RNA
(c) performing a reverse transcriptase reaction on said RNA using one or more of the primers selected from the group consisting of SEQ ID NO:1, 2, 3, 4, 5, 6, 8, 9, and 11;
(d) isolating the reaction products from step (c);
(e) using the reaction products isolated in step (d) as a template for a polymerase chain reaction amplification using a primer pair from claim 15; and
(f) detecting the product or products of said polymerase chain reaction amplification of step (e), thereby identifying the presence of bacteria in said sample that are expressing chlorite dismutase for reducing the (per)chlorate content of said sample.

46. (canceled)

47. (canceled)

48. (canceled)

49. (canceled)

50. (canceled)

51. (canceled)

52. (canceled)

53. (canceled)

54. (canceled)

55. A library of primers for the detection of a cld gene from DPRB, said library comprising at least 6 primers derived from the sequences set forth in SEQ ID NO:1, 2, 3, 4, 5, 6, 8, 9, 10 and 11.
